(19) United States
(12) Patent Application Publication
(10) Pub. No.: US 2022/0298257 A1
(43) Pub. Date: Sep. 22, 2022

(54) ANTI-CD22 ANTIBODIES AND USES THEREOF

(71) Applicant: Elpis Biopharmaceuticals, Lexington, MA (US)

(72) Inventors: Yan CHEN, Lexington, MA (US); Jenna NGUYEN, Lexington, MA (US); Kehao ZHAO, Lexington, MA (US)

(21) Appl. No.: 17/636,834

(22) PCT Filed: Aug. 21, 2020
(86) PCT No.: PCT/US20/47479

§ 371 Date: Feb. 18, 2022

Related U.S. Application Data
(60) Provisional application No. 62/889,739, filed on Aug. 21, 2019.

(51) Int. Cl. C07K 16/28; A61P 35/00; G01N 33/68

(52) U.S. Cl. CPC C07K 16/2896; A61P 35/00; G01N 33/68; A61K 2039/505

(57) ABSTRACT

Disclosed herein are high affinity anti-CD22 antibodies and methods of using such for therapeutic and/or diagnostic purposes. Also provided herein are methods for producing such anti-CD22 antibodies.

### CROSS-REFERENCE TO RELATED APPLICATIONS

This application claims the benefit of priority to U.S. Provisional Patent Application No. 62/889,739, filed Aug. 21, 2019, which is hereby incorporated by reference in its entirety.

### BACKGROUND OF THE INVENTION

Cluster of differentiation 22 (CD22) is a member of the SIGLEC family of lectins. This molecule expresses at a high level on the surface of mature B cells as relative to immature B-cells. As an inhibitory receptor for B cell receptor (BCR) signaling, it plays a regulatory role in preventing over-activation of the immune system.

It has been shown that CD22 is a promising target for leukemia treatment, such as acute lymphoplastic leukemia treatment, and for treatment of systemic autoimmune diseases.

### SUMMARY OF THE INVENTION

The present disclosure is based, at least in part, on the development of superior anti-CD22 antibodies having high binding affinity and specificity to CD22 expressed on cell surface. The anti-CD22 antibodies disclosed herein bind different CD22 epitopes as M971 and RFB4 (from which BL22 was derived), known anti-CD22 antibodies currently in pre-clinical and clinical studies. Further, certain exemplary anti-CD22 antibodies in IgG form (e.g., clone EP160-D02) showed high binding affinity and specificity to cell surface CD22, and higher ADCC activity relative to BL22 and M971. The results provided herein indicate that the anti-CD22 antibodies disclosed herein would be expected to have high therapeutic effects against CD22+ disease cells such as cancer cells.

Accordingly, one aspect of the present disclosure features an isolated antibody that binds CD22. Such anti-CD22 antibodies may bind to the same epitope as a reference antibody or competes against the reference antibody from binding to CD22. Exemplary reference antibody include EP35-A7, EP35-B5, EP35-C6, EP35-C6, EP35-C8, EP35-D6, EP35-E6, EP35-E7, EP97-A01, EP97-A10, EP97-B03, EP97-F01, EP97-G05, EP160-007, EP160-D02, EP160-E03, EP160-F04, EP160-F10, EP160-G04, EP160-G05, and EP160-H02, structural information of which is provided below. In specific examples, the reference antibody is EP160-D02. In other specific examples, the reference antibody is EP97-B03.

In some embodiments, the anti-CD22 antibody disclosed herein may comprise: (a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or (b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody. In some instances, the anti-CD22 antibody may have a binding affinity of less than 10 nm (e.g., less than 1 nM) to CD22 expressed on cell surface.

In some embodiments, the anti-CD22 antibody disclosed herein may comprise HC CDRs, which collectively contain no more than 8 amino acid residue variations as compared with the HC CDRs of the reference antibody; and/or LC CDRs of the antibody collectively contain no more than 8 amino acid residue variations as compared with the LC CDRs of the reference antibody.

Any of the anti-CD22 antibodies disclosed herein may comprise a V_{H} that is at least 85% identical to the V_{H} of the reference antibody, and/or a V_{L} that is at least 85% identical to the V_{L} of the reference antibody. In some examples, the anti-CD22 antibody may comprise the same heavy chain complementary determining regions (HC CDRs) and the same light chain complementary determining regions (LC CDRs) as the reference antibody. In particular examples, the anti-CD22 antibody may comprise the same V_{H} and the same V_{L} as the reference antibody.

Any of the anti-CD22 antibodies disclosed herein may be a human antibody or a humanized antibody. Alternatively or in addition, the anti-CD22 antibody may be a full-length antibody or an antigen-binding fragment thereof. In some examples, the anti-CD22 antibody is a single-chain antibody (scFv), for example, comprising the amino acid sequence of any one of SEQ ID NOs: 40-59.

In another aspect, provided herein is a nucleic acid or a set of nucleic acids, which collectively encodes the heavy chain and/or light chain of any of the anti-CD22 antibodies disclosed herein. In some embodiments, the nucleic acid or the set of nucleic acids can be a vector or a set of vectors, for example, expression vector(s). Also within the scope of the present disclosure are host cells (e.g., mammalian cells or bacterial cells) comprising any of the nucleic acid or the set of nucleic acids as disclosed herein, as well as pharmaceutical compositions comprising any of the anti-CD22 antibodies, any of the nucleic acid(s) encoding such, and host cells comprising the nucleic acid(s), and a pharmaceutically acceptable carrier.

Further, the present disclosure provides a method for inhibiting CD22 in a subject, comprising administering to a subject in need thereof any effective amount of the pharmaceutical composition as disclosed herein. In some embodiments, the subject can be a human patient having CD22 positive disease cells. For example, the subject may be a human patient having cancers or an autoimmune diseases, or other diseases/disorders involving CD22+ cells. Such a human patient may have CD22 positive cancer cells (e.g., hematopoietic cancer cells) or CD22 positive auto-reactive immune cells. Also within the scope of the present disclosure are pharmaceutical compositions as disclosed herein for use in treating a disease comprising CD22⁺ disease cells such as those described herein, as well as use of any of the anti-CD22 antibodies disclosed herein for manufacturing a medicament for use in treating any of the target diseases as also disclosed herein.

Moreover, the present disclosure provides a method for detecting presence of CD22, comprising: (i) contacting an antibody of any one of claims 1-12 with a sample suspected of containing CD22, and (ii) detecting binding of the antibody to CD22. The antibody may be conjugated to a detectable label. In some instances, the CD22 is expressed on cell surface. In some examples, the contacting step can be performed by administering the antibody to a subject.

In yet another aspect, the present disclosure provides a method of producing an antibody binding to CD22, comprising: (i) culturing the host cell of claim 16 under conditions allowing for expression of the antibody that binds CD22; and (ii) harvesting the antibody thus produced from the cell culture.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is an illustrative diagram showing an exemplary strategy for enriching high affinity CD22 binders from antibody libraries such as scFv libraries and single heavy chain (V_{H}) libraries.

FIG. 2 is a diagram showing exemplary single-chain (scFv) CD22 binders obtained from scFv libraries via multiple rounds of mRNA display selections followed by ELISA screening of individual positive clones.

FIGS. 3A-3D include diagrams showing titration curves of exemplary anti-CD22 antibodies to K562 cells expressing surface CD22. FIG. 3A: clones EP-84-A6, EP84-F6, EP84-H7, and EP84-G12. FIG. 3B: clones EP97-A10 and EP97-D06. FIG. 3C: clones EP160-004,

EP160-F04, EP160-007, EP160-H02, EP160-D02, EP97-A10, EP97-B03, and EP97-G05. FIG. 3D: EP160-G04, EP160-G01, EP160-E03, EP160-F10, and EP160-G05.

FIG. 4 is a diagram showing binding activity of exemplary anti-CD22 antibodies as indicated to CD22-expressing K562 cells in the presence or absence of anti-CD22 antibody M971.

FIG. 5 is a chart showing anti-CD22 antibody binding activity to cells expressing recombinant or endogenous CD22. For each tested anti-CD22 scFv antibody tested, bars from left to right correspond to K562 cells, CD22 K562 cells, CD22 HEK293 cells, Daudi cells, and Raji cells.

FIG. 6 is a photo showing immunohistochemistry (IHC) staining of endogenous CD22-positive cells using exemplary anti-CD22 scFv EP097-G05.

FIG. 7A and 7B include diagrams showing epitope binning of exemplary anti-CD22 antibodies as compared with known anti-CD22 antibodies M971 and RFB4. FIG. 7A: epitope binning assay relative to the M971 antibody. FIG. 7B: epitope binning relative to BL22, which is derived from the RFB4 antibody.

FIGS. 8A-8C include diagrams showing binding activity and specificity of anti-CD22 antibodies in IgG format. FIG. 8A a diagram showing the result of a binding assay using HEK293 cells expressing surface CD22. FIG. 8B a diagram showing the result of a binding assay using CHO-K1 cells expressing surface CD123. FIG. 8C a diagram showing the results of a binding activity as measured by ELISA.

FIG. 9 is a diagram showing antibody-dependent cellular cytotoxicity (ADCC) activity of anti-CD22 IgG antibodies as indicated.

FIG. 10 is a diagram showing internalization of anti-CD22 IgG antibodies as indicated after binding to cell surface CD22.

### DETAILED DESCRIPTION OF THE INVENTION

Provided herein are antibodies capable of binding to human CD22 (“anti-CD22 antibodies”), particularly CD22 expressed on cell surface. The anti-CD22 antibodies disclosed herein show high binding affinity to CD22 (e.g., cell-surface CD22), high stability, and/or bind to different CD22 epitopes as M971, a fully human anti-CD22 known in the art.

CD22 is a transmembrane glycoprotein expressed primarily on mature B cell surfaces. This cell surface receptor specifically binds sialic acid via an immunoglobulin (Ig) domain located at the N-terminus of the receptor. CD22 functions as an inhibitory receptor for the signaling pathway mediated by BCR. CD22 molecules from various species are well known in the art. For example, the amino acid sequence of human CD22 can be found under GenBank accession no. NP_001762.2.

CD22 is present on malignant B cells and thus is a promising target for treating hematopoietic cancer, particularly hematopoietic cancers of B cell origin, for example, acute lymphoblastic leukemia (ALL), B-cell non-Hodgkin's lymphoma (NHL) and chronic lymphocytic leukemia (CLL). CD22 is also involved in autoimmunity and thus would be a target for treating autoimmune diseases.

Thus, the anti-CD22 antibodies disclosed herein can serve as therapeutic agents for treating diseases having CD22+ disease cells, for example, cancers of B-cell linage or autoimmune diseases mediated by CD22⁺ auto-reactive immune cells. In addition, the anti-CD22 antibodies disclosed herein can serve as diagnostic agents for detecting presence of CD22, e.g., CD22-positive cells. The antibodies disclosed herein may also be used for research purposes.

### I. Antibodies Binding to CD22

The present disclosure provides antibodies binding to CD22, for example, human CD22. In some embodiments, the anti-CD22 antibodies disclosed herein are capable of binding to CD22 expressed on cell surface. As such, the antibodies disclosed herein may be used for either therapeutic or diagnostic purposes to target CD22-positive cells (e.g., leukemia cells). As used herein, the term “anti-CD22 antibody” refers to any antibody capable of binding to a CD22 polypeptide (e.g., a CD22 polypeptide expressed on cell surface), which can be of a suitable source, for example, human or a non-human mammal (e.g., mouse, rat, rabbit, primate such as monkey, etc.).

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-CD22 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion (e.g., chimeric antigen receptor or CAR), humanized antibodies, chimeric antibodies, diabodies, single domain antibody (e.g., a V_{H} only antibody such as a nanobody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

The anti-CD22 antibody described herein may be a full-length antibody, which contains two heavy chains and two light chains, each including a variable domain and a constant domain. Alternatively, the anti-CD22 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-CD22 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-CD22 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-CD22 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-CD22 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-CD22 antibodies described herein specifically bind to the corresponding target antigen (e.g., CD22) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (CD22) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e.., only baseline binding activity can be detected in a conventional method). In some examples, the anti-CD22 antibody disclosed herein does not bind to the same epitope as FMC63. In other examples, the anti-CD22 antibody binds to a CD22 epitope that is not overlapping with the CD22 epitope to which M971 binds. The V_{H} and V_{L} sequences of M971 are well known in the art and provided below (CDRs in boldface):

M971-V_{H} (SEQ ID NO: 1):
QVQLQQSGPGLVKPSQTLSLTCAISGDSVSSNSAAWNWIRQSPSRGLEW
LGRTYYRSWYNDYAVSVKSRITINPDTSKNQFSLQLNSVTPEDTAVYYC
AREVTGDLEDAFDIWGQGTMVTVSS
M971-V_{L} (SEQ ID NO: 2):
DIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTF
GQGTKLEIK

In some embodiments, an anti-CD22 antibody as described herein has a suitable binding affinity for the target antigen (e.g., CD22) or antigenic epitopes thereof. As used herein, “binding affinity” refers to the apparent association constant or K_{A}. The K_{A} is the reciprocal of the dissociation constant (K_{D}). The anti-CD22 antibody described herein may have a binding affinity (K_{D}) of at least 100 nM, 10 nM, 1 nM, 0.1 nM, or lower for CD22. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-CD22 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-CD22 antibody disclosed herein has an EC₅₀ value of lower than 10 nM, e.g., <1 nM, <0.5 nM, or lower than 0.1 nM, for binding to CD22-positive cells. As used herein, EC₅₀ values refer to the minimum concentration of an antibody required to bind to 50% of the cells in a CD22-positive cell population. EC₅₀ values can be determined using conventional assays and/or assays disclosed herein. See, e.g., Examples below.

A number of exemplary anti-CD22 antibodies are provided below (CDRs indicated in boldface as determined by the Chothia approach (Chothia et al. (1992) J. Mol. Biol., 227, 776-798, Tomlinson et al. (1995) EMBO J., 14, 4628-4638 and Williams et al.(1996) J. Mol. Biol., 264, 220-232). See also www2.mrc-lmb.cam.ac.uk/vbase/alignments2.php.

EP160-C07
V_{H} (SEQ ID NO: 3):
QMQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDAVAGSRGYWGQGTLVTVSS
V_{L} (SEQ ID NO: 4):
EIVLTQSPATLSVSPGEGVTLSCRASQSVSSNLAWYQQRPGQAPRLLIYGASIKATDVPDRFSGGGSGTD
FTLSISNLQSEDFAVYYCQQYHTWPPVTFGEGTKVEIK
EP160-E03
V_{H} (SEQ ID NO: 5):
EVQLVQSGGGVVQPGKSLRLSCAASGFTFSSYGMHWVRQAPGKGLEWVAVIWYDGSNKYYADSVKGRFTI
SRDNSKNTLYLQMNSLRAEDTAVYYCARDGWTGFDYWGQGTTVTVSS
V_{L} (SEQ ID NO: 6)
EIVLTQSPATLSVSPGEGVTLSCRASQSVSSNLAWYQQRPGQAPRLLIYGASIKATDVPDRFSGGGSGTD
FTLSISNLQSEDFAVYYCQQYHTWPPVTFGGGTKVEIK
EP160-F10 (a single domain antibody)
VH (SEQ ID NO: 7)
EVQLVESGGGVVQPGRSLRLSCVASGFTFRNYGMQWVRQTPDKGLEWVAVTAHDGTVQYYVDSVKGRFTI
SRDNSKDTLYLQMNSLRVADTAVYYCAKEATPRAADHFDYWGQGTLGTVSS
EP97410
V_{H} (SEQ ID NO: 8)
QVQLVQSGAEVKRPGASVKVSCKASGYTFTSYGISWVRQAPGQGLEWMGWISAYNGNTNYAQKLQGRVTM
TTDTSTSTAYMELRSLRSDDTAVYYCARDPGIAVAGTVDYWGQGTLVTVSS
V_{L} (SEQ ID NO: 9)
EIVMTQSPATLSVSPGEGVTLSCRASQSVSSNLAWYQQRPGQAPRLLIYGASIKATDVPDRFSGGGSGTD
FTLSISNVQSEDFAVYYCQQYHTWTPVTFGGGTKVEIK
EP97-1603
V_{H} (SEQ ID NO: 10)
QLVQSGAEVKKPGASVKVSCKASGYTFSSYGITWVRQAPGQGLEWMGWISAYNGNTNYAQKFQGRVTLTT
DTSTSIAYMELRSLTSDDTAVYYCATGGQEDYWGQGTLVTVSS
V_{L} (SEQ ID NO: 11)
EIVLTQSPATLSVSPGERATLSCRASQSVSSNLAWYQQKPGQAPRLLIYGASTRATGIPARFSGSGSGTE
FTLTISSLQSEDFAVYYCQQYNSWPPLTFGGGTKVEIK
EP160-D02
V_{H} (SEQ ID NO: 12)
QVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDPLEPLESDYWGQGTLVTVSS
V_{L} (SEQ ID NO: 13)
EIVMTQSPATLSVSPGEGVTLSCRASQSVSSNLAWYQQRPGQAPRLLIYGASIKATDVPDRFSGGGSGTD
FSLSITNLQSEDFAVYYCQQYHTWPPVTFGGGTKVEIK
EP160-G04
V_{H} (SEQ ID NO: 14)
QVQLVQSGAEVKKPGASVKVSCKASGYTFSSYGITWVRQAPGQGLEWMGWISAYNGNTNYAQKFQGRVTL
TTDTSTSIAYMELRSLTSDDTAVYYCATGGQEDYWGQGTLVTVSS
V_{L} (SEQ ID NO: 15)
EIVMTQSPATLSVSPGERATLSCRASQSVSSNLAWYQQKPGQAPRLLIYDASTRATGIPARFSGSGSGTE
FTLTISSLQSEDFAVYYCQQYHNWAPLTFGGGTKVGIK
EP160-H02
V_{H} (SEQ ID NO: 16)
QVQLVQSGAEVKKPGASVKVSCKASGYTFTSYGISWVRQAPGQGLEWMGGIIAYNGNTNYAQKLQGRVTM
TTDTSTSTAYMELRSLRSDDTAVYYCARDPPEYSSSAGTDYWGQGTLVTVSS
V_{L} (SEQ ID NO: 17)
EIVMTQSPATLSVSPGEGVTLSCRASQSVSSNLAWYQQRPGQAPRLLIYGASIKATDVPDRFSGGGSGTD
FTLSITNLQSEDFAVYYCQQYHTWSPVTFGGGTKVEIK
EP160-G05
V_{H} (SEQ ID NO: 18)
EVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDPSMDVWGQGTTVTVSS
V_{L} (SEQ ID NO: 19)
EIVLTQSPATLSVSPGERATLSCRASQSVSSNLAWYQQKPGQAPRLLIYGASTRATGIPARFSGSGSGTE
FTLTISSLQSEDFAVYYCQQYNSWPPITFGQGTRLEIK
EP35-C6
V_{H} (SEQ ID NO: 20)
QVQLVESGGGVVQPGGSLRLSCAASGFPFSRFGIHWVRQAPGKGLDWVAFIRTDGGSQHYADSVKGRFTI
SRDNSENMVYLQMNSLRVDDTALYYCAKDPPRVTGNTGYDYDWGQGVQVTVSS
V_{L} (SEQ ID NO: 21)
DIVMTQSPDSLAVSLGERATINCKSSQSVLYSANNKNCLAWYQQKSGQPPKLLIYWASTRESGVPGRFSG
SGSGTDFTLTISSLQAEDVAVYYCQQYYSPPRTFGQGTKLEIK
EP35-A7
V_{H} (SEQ ID NO: 22)
EVQLVESRGGVVQPGGSLRLSCAASGFTFSSYGMHWVRQAPGKGLEWVAFIRYDGSNKYYADSVKGRFTI
SRDNSKNTLYLQMNSLRAEDTAVYYCAKETVTTNYYYYMDVWGKGTTVTVSS
V_{L} (SEQ ID NO: 23)
DVVMTQSPLSLPVTLGQPASISCRSSRSLEYNDGNTYLNWFHQRPGQSPRRLIYKVSNRDSGVPDRFSGS
GSDTDFTLKISRVEAEDVGIYYCMQGTHWPLTFGQGTRLEIK
EP35-D6
V_{H} (SEQ ID NO: 24)
QVQLVQSGTEVKKPGASVKVSCKASGYTFTNNAITWVRQAPGQGLEWMGYISTSSDNINYAQKFRGRLTL
TTDTSTGTAYMELSSLRSDDTATYYCARDGIFGGRDDPWGQGTLVTVSS
V_{L} (SEQ ID NO: 25)
DIVMTQSPSSLSASVGDRVTITCQASQDISNYLNWYQQKPGKAPKLLIYDASNLETGVPSRFSGSGSETD
FTITISSLQPEDIATYYCQQYDNLPLTFGGGTKVR
EP35-E6
V_{H} (SEQ ID NO: 26)
QVQLVESGGALVQPGGSLRLSCVVSGFPFSTAWMNWVRQAPGKGLEWVARIKSEAHGGTTHYAPPVQGRF
TISRDDSKNTVSLQMNSLKTEDTGVYYCGDFQWGQGTLVTVSS
V_{L} (SEQ ID NO: 27)
VIWMTQSPSSLSASVGDRITITCQASQDISNFLNWYQQKPGEAPKLLLYDASNLERGVPSRFSGGGSGTD
FTLTISSLQPEDIATYFCQQYDNLPLTFGGGTKVEIK
EP35-C8
V_{H} (SEQ ID NO: 28)
QVQLVQSGAEVKKPGASVKVSCKASGYTFTSYGISWVRQAPGQGLEWMGWISAYNGNTNYAQKLQGRVTM
TTDTSTSTAYMELRSLGSDDTAVYYCARDSGSSDLDYWGQGTLVTVSS
V_{L} (SEQ ID NO: 29)
EIVMTQSPATLSVSPGEGVTLSCRASQSVSSNLAWYQQKPGQAPRLLMYGASIKATDVPDRFSGGGSGTD
FTLSISSLQSEDFAVYYCQQYHTWPPVTFGGGTKVEIK
EP160-F04
V_{H} (SEQ ID NO: 30)
EVQLVQSGAEVKKPGASVKVSCKASGYTFTSYGISWVRQAPGQGLEWMGWISAYNGNTNYAQKLQGRVTM
TTDTSTSTAYMELRSLKSDDTAVYYCAISIGAFDIWGQGTMVTVSS
V_{L} (SEQ ID NO: 31)
EIVMTQSPATLSVSPGEEVTLSCRASQSVSSNLAWYQQRPGQAPRLLIYGASIKATDVPDRFSGGGSGTD
FTLSISNLQSEDFAVYYCQQYHTWPPVTFGGGTKVEIK
BP35-1605
V_{H} (SEQ ID NO: 32)
QVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDSGNSPIDYWGQGTLVTVSS
V_{L} (SEQ ID NO: 33)
EIVMTQSPATLSVSPGEGVTLSCRASQSVSSNLAWYQQRPGQAPRLLIYGASIKATDVPDRFSGGGSGTD
FTLSISNLQSEDFAVYYCQQYHTWPPVTFGGGTKVEIK
EP97-G05
V_{H} (SEQ ID NO: 34)
EVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDYGDPSGDDYWGQGTLVTVSS
V_{L} (SEQ ID NO: 35)
EIVLTQSPATLSVSPGEGVTLSCRASQSVSSNLAWYQQRPGQAPRLLIYGASIKATDVPDRFSGGGSGTD
FTLSISNLQSEDFAVYYCQQYHTWPPVTFGGGTKVEIK
EP97-F01
V_{H} (SEQ ID NO: 36)
QVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDHIAAAGDYWGQGTLVTVSS
V_{L} (SEQ ID NO: 37)
EIVMTQSPATLSVSPGEGVTLSCRASQSVSSNLAWYQQRPGQAPRLLIYGASIKATDVPDRFSGGGSGTD
FTLSITNLQSEDFAVYYCQQYHTWPPVTFGGGTKVEIK
EP97401
V_{H} (SEQ ID NO: 38)
EVQLVQSGGGVVQPGRSLKLSCAASGFTFSSYGMHWVRQAPGKGLEWVAVIWYDGSNKYYADSVKGRFTI
SRDNSKNTLYLQMNSLRAEDTAVYYCARDGWKGFDYWGQGTTVTVSS
V_{L} (SEQ ID NO: 39)
EIVLTQSPATLSVSPGEGVTLSCRASQSVSSNLAWYQQRPGQAPRLLIYGASIKATDVPDRFSGGGSGTD
FTLSISNLQSEDFAVYYCQQYHTWPPVTFGGGTKVEIK

In some embodiments, the anti-CD22 antibodies described herein bind to the same epitope of a CD22 polypeptide as any of the exemplary antibodies described herein (for example, EP35-A7, EP35-B5, EP35-C6, EP35-C8, EP35-D6, EP35-E6, EP35-E7, EP97-A01, EP97-A10, EP97-B03, EP97-F01, EP97-G05, EP160-007, EP160-D02, EP160-E03, EP160-F04, EP160-F10, EP160-G04, EP160-G05, EP160-H02, and EP97-A01) or compete against the exemplary antibody from binding to the CD22 antigen. In some examples, the anti-CD22 antibodies disclosed herein bind to the same epitope of a CD22 polypeptide as EP160-D02 or compete against the exemplary antibody from binding to the CD22 antigen. In other examples, the anti-CD22 antibodies disclosed herein bind to the same epitope of a CD22 polypeptide as EP97-B03 or compete against the exemplary antibody from binding to the CD22 antigen.

An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some examples, the anti-CD22 antibody comprises the same V_{H} and/or V_{L} CDRs as an exemplary antibody described herein. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-CD22 antibodies may have the same V_{H}, the same V_{L}, or both as compared to an exemplary antibody described herein.

Also within the scope of the present disclosure are functional variants of any of the exemplary anti-CD22 antibodies as disclosed herein (e.g., EP160-D2 or EP97-B03). Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope of CD22 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an anti-CD22 antibody may comprise a V_{H} fragment having CDR amino acid residue variations in only the heavy chain CDR1 as compared with the V_{H} of the exemplary antibody. In some examples, the anti-CD22 antibody may further comprise a V_{L} fragment having the same V_{L} CDR3, and optionally same V_{L} CDR1 or V_{L} CDR₂ as the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-CD22 antibody may comprise heavy chain CDRs that are at least 80% (e.g., 85%, 90%, 95%, or 98%) sequence identity, individually or collectively, as compared with the V_{H} CDRs of an exemplary antibody described herein. Alternatively or in addition, the anti-CD22 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} or V_{L} CDRs of an antiody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-CD22 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-CD22 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the anti-CD22 antibody disclosed herein may be a single chain antibody (scFv). A scFv antibody may comprise a V_{H} fragment and a V_{L} fragment, which may be linked via a flexible peptide linker. In some instances, the scFv antibody may be in the V_{H}→V_{L} orientation (from N-terminus to C-terminus). In other instances, the scFv antibody may be in the V_{L}→V_{H} orientation (from N-terminus to C-terminus). Exemplary scFv anti-CD22 antibodies are provided below (CDRs in boldface and peptide linker in boldface and underlined):

EP160-007 (scFv, V_{H}-V_{L} orientation; SEQ ID NO: 40)
QMQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDAVAGSRGYWGQGTLVTVSSGGGGSGGGGSGGGGSEIVLTQS
PATLSVSPGEGVTLSCRASQSVSSNLAWYQQRPGQAPRLLIYGASIKATDVPDRFSGGGSGTDFTLSISN
LQSEDFAVYYCQQYHTWPPVTFGEGTKVEIK
EP160-E03 (scFv, V_{H}-V_{L} orientation; SEQ ID NO: 41)
EVQLVQSGGGVVQPGKSLRLSCAASGFTFSSYGMHWVRQAPGKGLEWVAVIWYDGSNKYYADSVKGRFTI
SRDNSKNTLYLQMNSLRAEDTAVYYCARDGWTGFDYWGQGTTVTVSSGGGGSGGGGSGGGGSEIVLTQSP
ATLSVSPGEGVTLSCRASQSVSSNLAWYQQRPGQAPRLLIYGASIKATDVPDRFSGGGSGTDFTLSISNL
QSEDFAVYYCQQYHTWPPVTFGGGTKVEIK
EP160-F10 (single chain antibody; SEQ ID NO: 42)
EVQLVESGGGVVQPGRSLRLSCVASGFTFRNYGMQWVRQTPDKGLEWVAVTAHDGTVQYYVDSVKGRFTI
SRDNSKDTLYLQMNSLRVADTAVYYCAKEATPRAADHFDYWGQGTLGTVSS
EP97-A10 (scFv, V_{H}-V_{L} orientation; SEQ ID NO: 43)
QVQLVQSGAEVKRPGASVKVSCKASGYTFTSYGISWVRQAPGQGLEWMGWISAYNGNTNYAQKLQGRVTM
TTDTSTSTAYMELRSLRSDDTAVYYCARDPGIAVAGTVDYWGQGTLVTVSSGGGGSGGGGSGGGGSEIVM
TQSPATLSVSPGEGVTLSCRASQSVSSNLAWYQQRPGQAPRLLIYGASIKATDVPDRFSGGGSGTDFTLS
ISNVQSEDFAVYYCQQYHTWTPVTFGGGTKVEIK
EP97-B03 (scFv, V_{H}-V_{L} orientation; SEQ ID NO: 44)
QLVQSGAEVKKPGASVKVSCKASGYTFSSYGITWVRQAPGQGLEWMGWISAYNGNTNYAQKFQGRVTLTT
DTSTSIAYMELRSLTSDDTAVYYCATGGQEDYWGQGTLVTVSSGGGGSGGGGSGGGGSEIVLTQSPATLS
VSPGERATLSCRASQSVSSNLAWYQQKPGQAPRLLIYGASTRATGIPARFSGSGSGTEFTLTISSLQSED
FAVYYCQQYNSWPPLTFGGGTKVEIK
EP160-D02 (scFv, V_{H}-V_{L} orientation; SEQ ID NO: 45)
QVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDPLEPLESDYWGQGTLVTVSSGGGGSGGGGSGGGGSEIVMTQ
SPATLSVSPGEGVTLSCRASQSVSSNLAWYQQRPGQAPRLLIYGASIKATDVPDRFSGGGSGTDFSLSIT
NLQSEDFAVYYCQQYHTWPPVTFGGGTKVEIK
EP160-G04 (scFv, V_{H}-V_{L} orientation; SEQ ID NO: 46)
QVQLVQSGAEVKKPGASVKVSCKASGYTFSSYGITWVRQAPGQGLEWMGWISAYNGNTNYAQKFQGRVTL
TTDTSTSIAYMELRSLTSDDTAVYYCATGGQEDYWGQGTLVTVSSGGGGSGGGGSGGGGSEIVMTQSPAT
LSVSPGERATLSCRASQSVSSNLAWYQQKPGQAPRLLIYDASTRATGIPARFSGSGSGTEFTLTISSLQS
EDFAVYYCQQYHNWAPLTFGGGTKVGIK
EP160-H02 (scFv, V_{H}-V_{L} orientation; SEQ ID NO: 47)
QVQLVQSGAEVKKPGASVKVSCKASGYTFTSYGISWVRQAPGQGLEWMGGIIAYNGNTNYAQKLQGRVTM
TTDTSTSTAYMELRSLRSDDTAVYYCARDPPEYSSSAGTDYWGQGTLVTVSSGGGGSGGGGSGGGGSEIV
MTQSPATLSVSPGEGVTLSCRASQSVSSNLAWYQQRPGQAPRLLIYGASIKATDVPDRFSGGGSGTDFTL
SITNLQSEDFAVYYCQQYHTWSPVTFGGGTKVEIK
EP160-G05 (scFv, V_{H}-V_{L} orientation; SEQ ID NO: 48)
EVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDPSMDVWGQGTTVTVSSGGGGSGGGGSGGGGSEIVLTQSPAT
LSVSPGERATLSCRASQSVSSNLAWYQQKPGQAPRLLIYGASTRATGIPARFSGSGSGTEFTLTISSLQS
EDFAVYYCQQYNSWPPITFGQGTRLEIK
EP35-F7 (Identical to EP97-A01, (scFv, V_{H}-V_{L} orientation; SEQ ID NO: 49)
EVQLVQSGGGVVQPGRSLRLSCAASGFTFSSYGMHWVRQAPGKGLEWVAVIWYDGSNKYYADSVKGRFTI
SRDNSKNTLYLQMNSLRAEDTAVYYCARDGWKGFDYWGQGTTVTVSSGGGGSGGGGSGGGGSEIVLTQSP
ATLSVSPGEGVTLSCRASQSVSSNLAWYQQRPGQAPRLLIYGASIKATDVPDRFSGGGSGTDFTLSISNL
QSEDFAVYYCQQYHTWPPVTFGGGTKVEIK
EP35-C6 (scFv, V_{H}-V_{L} orientation; SEQ ID NO: 50)
QVQLVESGGGVVQPGGSLRLSCAASGFPFSRFGIHWVRQAPGKGLDWVAFIRTDGGSQHYADSVKGRFTI
SRDNSENMVYLQMNSLRVDDTALYYCAKDPPRVTGNTGYDYDWGQGVQVTVSSGGGGSGGGGSGGGGSDI
VMTQSPDSLAVSLGERATINCKSSQSVLYSANNKNCLAWYQQKSGQPPKLLIYWASTRESGVPGRFSGSG
SGTDFTLTISSLQAEDVAVYYCQQYYSPPRTFGQGTKLEIK
EP35-A7 (scFv, V_{H}-V_{L} orientation; SEQ ID NO: 51)
EVQLVESRGGVVQPGGSLRLSCAASGFTFSSYGMHWVRQAPGKGLEWVAFIRYDGSNKYYADSVKGRFTI
SRDNSKNTLYLQMNSLRAEDTAVYYCAKETVTTNYYYYMDVWGKGTTVTVSSGGGGSGGGGSGGGGSDVV
MTQSPLSLPVTLGQPASISCRSSRSLEYNDGNTYLNWFHQRPGQSPRRLIYKVSNRDSGVPDRFSGSGSD
TDFTLKISRVEAEDVGIYYCMQGTHWPLTFGQGTRLEIK
EP35-D6 (scFv, V_{H}-V_{L} orientation; SEQ ID NO: 52)
QVQLVQSGTEVKKPGASVKVSCKASGYTFTNNAITWVRQAPGQGLEWMGYISTSSDNINYAQKFRGRLTL
TTDTSTGTAYMELSSLRSDDTATYYCARDGIFGGRDDPWGQGTLVTVSSGGGGSGGGGSGGGGSDIVMTQ
SPSSLSASVGDRVTITCQASQDISNYLNWYQQKPGKAPKLLIYDASNLETGVPSRFSGSGSETDFTITIS
SLQPEDIATYYCQQYDNLPLTFGGGTKVR
EP35-E6 (scFv, V_{H}-V_{L} orientation; SEQ ID NO: 53)
QVQLVESGGALVQPGGSLRLSCVVSGFPFSTAWMNWVRQAPGKGLEWVARIKSEAHGGTTHYAPPVQGRF
TISRDDSKNTVSLQMNSLKTEDTGVYYCGDFQWGQGTLVTVSSGGGGSGGGGSGGGGSVIWMTQSPSSLS
ASVGDRITITCQASQDISNFLNWYQQKPGEAPKLLLYDASNLERGVPSRFSGGGSGTDFTLTISSLQPED
IATYFCQQYDNLPLTFGGGTKVEIK
EP35-C8 (scFv, V_{H}-V_{L} orientation; SEQ ID NO: 54)
QVQLVQSGAEVKKPGASVKVSCKASGYTFTSYGISWVRQAPGQGLEWMGWISAYNGNTNYAQKLQGRVTM
TTDTSTSTAYMELRSLGSDDTAVYYCARDSGSSDLDYWGQGTLVTVSSGGGGSGGGGSGGGGSEIVMTQS
PATLSVSPGEGVTLSCRASQSVSSNLAWYQQKPGQAPRLLMYGASIKATDVPDRFSGGGSGTDFTLSISS
LQSEDFAVYYCQQYHTWPPVTFGGGTKVEIK
EP160-F04 (scFv, V_{H}-V_{L} orientation; SEQ ID NO: 55)
EVQLVQSGAEVKKPGASVKVSCKASGYTFTSYGISWVRQAPGQGLEWMGWISAYNGNTNYAQKLQGRVTM
TTDTSTSTAYMELRSLKSDDTAVYYCAISIGAFDIWGQGTMVTVSSGGGGSGGGGSGGGGSEIVMTQSPA
TLSVSPGEEVTLSCRASQSVSSNLAWYQQRPGQAPRLLIYGASIKATDVPDRFSGGGSGTDFTLSISNLQ
SEDFAVYYCQQYHTWPPVTFGGGTKVEIK
EP35-B05 (scFv, V_{H}-V_{L} orientation; SEQ ID NO: 56)
QVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDSGNSPIDYWGQGTLVTVSSGGGGSGGGGSGGGGSEIVMTQS
PATLSVSPGEGVTLSCRASQSVSSNLAWYQQRPGQAPRLLIYGASIKATDVPDRFSGGGSGTDFTLSISN
LQSEDFAVYYCQQYHTWPPVTFGGGTKVEIK
EP97-G05 (scFv, V_{H}-V_{L} orientation; SEQ ID NO: 57)
EVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDYGDPSGDDYWGQGTLVTVSSGGGGSGGGGSGGGGSEIVLTQ
SPATLSVSPGEGVTLSCRASQSVSSNLAWYQQRPGQAPRLLIYGASIKATDVPDRFSGGGSGTDFTLSIS
NLQSEDFAVYYCQQYHTWPPVTFGGGTKVEIK
EP97-F01 (scFv, V_{H}-V_{L} orientation; SEQ ID NO: 58)
QVQLVQSGAEVKKPGASVKVSCKASGYTFTSYGISWVRQAPGQGLEWMGWISAYNGNTNYAQKLQGRVTM
TTDTSTSTAYMELRSLRSDDTAVYYCARDHIAAAGDYWGQGTLVTVSSGGGGSGGGGSGGGGSEIVMTQS
PATLSVSPGEGVTLSCRASQSVSSNLAWYQQRPGQAPRLLIYGASIKATDVPDRFSGGGSGTDFTLSITN
LQSEDFAVYYCQQYHTWPPVTFGGGTKVEIK
EP97-A01 (scFv, V_{H}-V_{L} orientation; SEQ ID NO: 59)
EVQLVQSGGGVVQPGRSLKLSCAASGFTFSSYGMHWVRQAPGKGLEWVAVIWYDGSNKYYADSVKGRFTI
SRDNSKNTLYLQMNSLRAEDTAVYYCARDGWKGFDYWGQGTTVTVSSGGGGSGGGGSGGGGSEIVLTQSP
ATLSVSPGEGVTLSCRASQSVSSNLAWYQQRPGQAPRLLIYGASIKATDVPDRFSGGGSGTDFTLSISNL
QSEDFAVYYCQQYHTWPPVTFGGGTKVEIK

Any of the anti-CD22 antibody as described herein, e.g., the exemplary anti-CD22 antibodies provided here such as EP160-D2 or EP97-B03, can bind and inhibit (e.g., reduce or eliminate) the activity of CD22-positive cells (e.g., B cells). In some embodiments, the anti-CD22 antibody as described herein can bind and inhibit the activity of CD22-positive cells by at least 30% (e.g., 35%, 40%, 45%, 50%, 60%, 70%, 80%, 90%, 95% or greater, including any increment therein). The inhibitory activity of an anti-CD22 antibody described herein can be determined by routine methods known in the art, e.g., by an assay for measuring the K_{i,}^{app} value.

In some examples, the K_{i,}^{app} value of an antibody may be determined by measuring the inhibitory effect of different concentrations of the antibody on the extent of a relevant reaction; fitting the change in pseudo-first order rate constant (ν) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{\begin{matrix}( [ E ] - [ I ] - {K}_{i}^{app} ) + \\ \sqrt{{( [ E ] - [ I ] - {K}_{i}^{app} )}^{2} + 4 [ E ]} \cdot {K}_{i}^{app}\end{matrix}}{( 2 )} \qquad ( Equation 1 )$$

Where A is equivalent to νₒ/E, the initial velocity (νₒ) of the enzymatic reaction in the absence of inhibitor (I) divided by the total enzyme concentration (E). In some embodiments, the anti-CD22 antibody described herein may have a Ki^{app} value of 1000, 500, 100, 50, 40, 30, 20, 10, 5 pM or less for the target antigen or antigen epitope.

### II. Preparation of Anti-CD22 Antibodies

Antibodies capable of binding CD22 as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-CD22 antibody may be identified from a suitable library (e.g., a human antibody library).

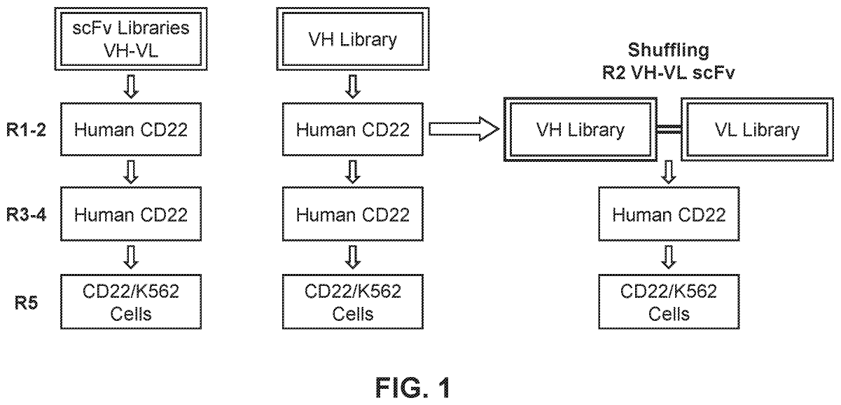

In some instances, high affinity fully human CD22 binders may be obtained from a human antibody library following the screening strategy illustrated in FIG. 1. See also Example 1 below. This strategy allows for maximizing the library diversity to cover board and active epitopes on CD22 expressing cells.

If desired, an antibody (monoclonal or polyclonal) of interest (e.g., produced by a hybridoma cell line or isolated from an antibody library) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively or in addition, it may be desirable to genetically manipulate the antibody sequence to obtain greater affinity and/or specificity to the target antigen and greater efficacy in enhancing the activity of CD22. It will be apparent to one of skill in the art that one or more polynucleotide changes can be made to the antibody and still maintain its binding specificity to the target antigen.

Alternatively, antibodies capable of binding to the target antigens as described herein (a CD22 molecule) may be isolated from a suitable antibody library via routine practice. Antibody libraries can be used to identify proteins that bind to a target antigen (e.g., human CD22 such as cell surface CD22) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage-display, yeast-display, mammalian cell-display, or mRNA-display scFv library and scFv clones specific to CD22 can be identified from the library following routine procedures. Positive clones can be subjected to further screening to identify those that enhance CD22 activity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of CD22 have been replaced (swapped) with sequences from a closely related, but antigenically distinct protein (such as another member of the tumor necrosis factor receptor family). By assessing binding of the antibody to the mutant CD22, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-CD22 antibody is prepared by recombinant technology as exemplified below.

Nucleic acids encoding the heavy and light chain of an anti-CD22 antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-CD22 antibody, as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-CD22 antibody and the other encoding the light chain of the anti-CD22 antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-CD22 antibody as described herein, vectors (e.g., expression vectors) containing such; and host cells comprising the vectors are within the scope of the present disclosure.

### III. Applications of Anti-CD22 Antibodies

Any of the anti-CD22 antibodies disclosed herein can be used for therapeutic, diagnostic, and/or research purposes, all of which are within the scope of the present disclosure.

### Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Therapeutic Applications

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying CD22⁺ disease cells. Examples of such target diseases/disorcers include hematopoietic cancers, e.g., a cancer of B cell lineage. Examples include, but are not limited to, hematological B cell neoplasms including lymphocytic leukemia, e.g., B Cell chronic lymphocytic leukemia (CLL); B-cell acute lymphoblastic leukemia (ALL), and B-cell non-Hodgkin's lymphoma (NHL). Alternatively, the CD22⁺ disease cells can be immune cells (e.g., B cells) specific to autoantigens.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject having a target autoimmune disease also can be identified by routine medical examinations. In some embodiments, the subject to be treated by the method described herein may be a human patient having an autoimmune disease. Such a human patient may have undergone or is undergoing a therapy for the autoimmune disease.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. In some examples, the dosage of the anti-CD22 antibody described herein can be 10 mg/kg. The particular dosage regimen, i.e.., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e.., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### Kits for Use in Treatment of Diseases

The present disclosure also provides kits for use in treating or alleviating a target disease, such as hematopoietic cancer as described herein. Such kits can include one or more containers comprising an anti-CD22 antibody, e.g., any of those described herein. In some instances, the anti-CD22 antibody may be co-used with a second therapeutic agent.

In some embodiments, the kit can comprise instructions for use in accordance with any of the methods described herein. The included instructions can comprise a description of administration of the anti-CD22 antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-CD22 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-CD22 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1

### Generation of Fully Human Anti-CD22 Antibodies

Fully human antibodies having binding specificity to cell-surface human CD22 were identified from a human antibody library as follows.

### Generation of CD22 Overexpression Recombinant Cell Lines

HEK293 and K562 cells (ATCC) were transfected with a pCMV6-Entry vector carrying a nucleotide sequence encoding the full-length human CD22 fused with flag and Myc tags at the C-terminus. G418 drug selection process yielded a polyclonal, drug resistant pool of CD22-expressing cells. In parallel, the parental cell line transferred with the empty pCMV6-Entry vector was generated for use as a negative control. The CD22-expressing cells were sorted by FACS to yield a pool of CD22-expressing cells. The pool was expanded under G418 drug selection. Single cell sorting was then performed followed by further drug selection to generate clonal cell lines. The clonal lines were screened for CD22 expression by FACS. The cell line showing a high expression level of CD22 was selected for use in selection, screening and assays as disclosed herein.

### Screening for Anti-CD22 Antibodies From a Human Antibody Libraries

Natural human antibody libraries were constructed from bone marrow MNCs and PBMCs of multiple naïve health donors and autoimmune disease patient doners. RT-PCR was performed to capture the full immunoglobulin repertoire of both V_{H} and V_{L} domains (producing V_{H} and V_{L} libraries). A single-chain antibody (scFv) library was then constructed by V_{H} and V_{L} shuffling. The library size is predicted to be 10¹²⁻¹³. The V_{H} and scFv libraries have been further modified to insert in vitro transcription and translation signals at the N-terminus and a flag tag to the C-terminus of the antibody fragment, respectively, for selection by mRNA display.

mRNA display technology was then used for the identification of CD22 binders from the above constructed V_{H} and scFv libraries following conventional practice (see, e.g., U.S. Pat. No. 6,258,558B1, the relevant disclosures of which are incorporated by reference herein for the subject matter or purpose referenced herein. Briefly, the DNA libraries were first transcribed into mRNA libraries and then translated into mRNA-V_{H} or scFv fusion libraries by covalent coupling through a puromycin linker. The libraries were then purified and converted to mRNA/cDNA fusion libraries. The fusion libraries were first counter selected with human IgGs (negative selections) or K562 cells to remove non-specific binders, followed by selection against either recombinant CD22-Fc fusion protein captured on Protein G magnetic beads (round 1-3) or on CD22 overexpression recombinant K562 cells (round 4). The CD22 binders were recovered and enriched by PCR amplification. At round 3, enriched V_{H} library was converted to scFv library by shuffling with a naïve V_{L} library noted above and further enriched for 3 more rounds. A total of 4 rounds of selections was executed to generate highly enriched anti-CD22 antibody pools, as illustrated in FIG. 1.

The enriched anti-CD22 antibody pools were cloned into the bacterial periplasmic expression vector pET22b, which was transformed into TOP 10 competent cells. Each of the scFv molecules was engineered to have a C-terminal flag and 6×HIS tag for purification and assay detection. Clones from TOP 10 cells were pooled and the miniprep DNA were prepared and subsequently transformed into bacterial Rosetta II strain for expression. Single clone was picked, grown and induced with 0.1 mM IPTG in 96 well plate for expression. The supernatant was collected after 16-24 hours induction at 30° C. for assays to identify anti-CD22 antibodies.

The supernatant samples were assessed with sandwich ELISA assay to determine the presence/level of the anti-CD22 scFv antibody contained therein. Briefly, a 96 well plate was immobilized with anti-HIS tag antibody (R&D Systems) at a final concentration of 2 μg/mL in 1×PBS in a total volume of 50 μL per well. The plate was incubated overnight at 4° C. followed by blocking with 200 μL per well of a superblock buffer for 1 hour. 100 μl of 1:10 1×PBST diluted supernatant were added to each well and incubated for 1 hour with shaking. The expression level of the CD22 scFv was detected by incubating the mixture in the plate with 50 μL of an HRP-conjugated anti-Flag antibody, which is diluted at 1:5000 in 1×PBST, for one hour. In between each step, the plate was washed 3 times with 1×PBST in plate washer. The plate was then developed with 50 μl of the TMB substrate for 5 mins and stopped by adding 50 μl of 2N sulfuric acid. The plate was read at OD450 nm in Biotek plate reader and the data was analyzed with Excel bar graph.

CD22 binding screening ELISA was developed to identify individual CD22 binders. Briefly, 96 well plate was immobilized with a human Fc as a control or a human CD22-Fc protein at final concentration of 2 μg/mL in 1×PBS in a total volume of 50 μL per well. The plate was incubated overnight at 4° C. followed by blocking with 200 μL per well of a superblock buffer for 1 hour. 100 μl of the supernatant was added to each of the Fc and CD22-Fc fusion protein immobilized wells and incubated for 1 hour with shaking. The CD22 binding was detected by adding a 50 μL of HRP-conjugated anti-Flag antibody, which was diluted at 1:5000 in 1×PBST. In between each step, the plate was washed 3 times with 1×PBST in a plate washer. The plate was then developed with 50 μl of the TMB substrate for 5 minutes and stopped by adding 50 μl of 2N sulfuric acid. The plate was read at OD450 nm Biotek plate reader and the binding and selectivity was analyzed with Excel bar graph.

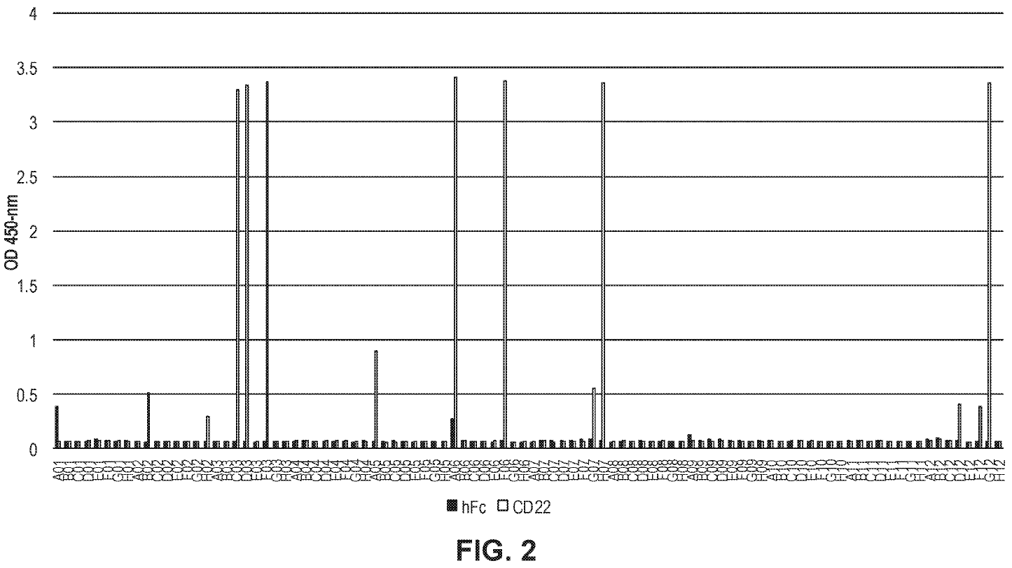

A number of positive anti-CD22 clones was identified in the screening process disclosed herein as exemplified in FIG. 2.

### EXAMPLE 2

### Identification of Exemplary Anti-CD22 Clones Capable of Binding to Cell Surface-Expressed CD22

### Production and Purification of Anti-CD22 Antibodies in E.coli Cells

Cells expressing V_{H} or anti-CD22 scFv antibodies identified in the screening process disclosed in Example 1 above were picked from a glycerol stock plate and grown overnight into a 5 mL culture in a Thomson 24-well plate with a breathable membrane. Bacterial cells as described in the Examples herein were grown at 37° C. and shaking at 225 RPM in Terrific Broth Complete plus 100 μg/mL carbenicillin and 34 μg/mL chloramphenicol, with 1:5,000 dilution of antifoam-204 also added, unless specified otherwise. The overnight starter culture was then used to inoculate a larger culture at a suitable dilution rate of starter culture into the designated production culture (e.g., 50 mL culture in 125 mL Thomson Ultra Yield flask, 100 mL culture in 250 mL Ultra Yield Thomson flask or 250 mL culture in 500 mL Ultra Yield Thomson flask) and grown until the OD₆₀₀ was between 0.5-0.8. At this point, the culture was induced with a final concentration of IPTG at 0.5 mM for V_{H} and 0.1 mM scFv and incubated over night at 30° C. The cultures were then spun for 30 min at 5,000×g, to pellet the cells and the supernatant was filter sterilized through a 0.2 μm sterilizing PES membrane for further analysis.

To purify the antibody fragment, 3 μl GE Ni Sepharose Excel resin were mixed with 1 mL of filtered supernatant and loaded onto 10 mL or 20 mL BioRad Econo-Pac columns. Before loading, the resin of the column was equilibrated with at least 20 column volume (CV) buffer A (1×PBS, pH7.4 with extra NaCl added to 500 mM). The filter sterilized supernatant was purified by gravity flow via either controlling the flow to 1 mL/min or being poured over two times, over the same packed resin bed. The column was then washed with the following buffers: 10 CV buffer A, 20 CV buffer B (1×PBS, pH7.4 with extra NaCl to 500 mM, and 30 mM imidazole). The two Detox buffers were used to remove endotoxin, if needed. To purify the antibody fragment from the 250 mL expression culture, antibody-bound column was washed sequentially with 20 CV buffer C (1×PBS pH7.4 with extra NaCl to 500 mM, 1% Tx114), 20 CV buffer D (1×PBS pH7.4 with extra NaCl to 500 mM, 1% Tx100+0.2% TNBP) and 40 CV buffer E (1×PBS pH7.4 with extra NaCl to 500 mM).

The protein was eluted with Eluting buffer F (1×PBS pH7.4 with extra NaCl to 500 mM, and 500 mM imidazole) in a total of six fractions (0.5 CV pre elute, 5×1 CV elute). Fractions were run on a Bradford assay (100 ul diluted Bradford solution+10 ul sample). Fractions with bright blue color were pooled and the protein concentration thereof was measured by A280 extension coefficient. SDS-PAGE gel assay was performed to analyze the purity of the purified antibodies.

In most cases, Tm shift thermal stability assay was performed to measure the thermal stability of the purified antibodies.

### Cell Surface Binding Activity of Anti-CD22 scFv Antibody by FACS Analysis

To determine the binding EC₅₀ value of each anti-CD22 antibody to cell surface-expressed CD22, each purified scFv protein was titrated from 100 nM with 2-fold serial dilutions in full medium. The diluted samples were incubated with CD22-expressing HEK293 cells (CD22/HEK293 cells) in 96 wells plate on ice for 1 hour. Cells were spun down at 1200 rpm for 5 minutes at 4° C. to remove unbound antibodies. Cells were then washed once with 200 μL of full medium per well. Samples were mixed with an Alexa fluor 488-conjugated anti-His antibody (secondary antibody, 100 μL, 1:1000 diluted) and incubated at 4° C. for 30 minutes in dark. Samples were then spun down at 1200 rpm for 5 minutes at 4° C. and washed twice with 200 uL of 1×PBS per well. The resultant samples were reconstituted in 200 uL of 1×PBS and read on Guava EasyCyte. Analysis was done by counting only Alexa Fluor 488-positive cells and then plotted in Prism 8.1 software.

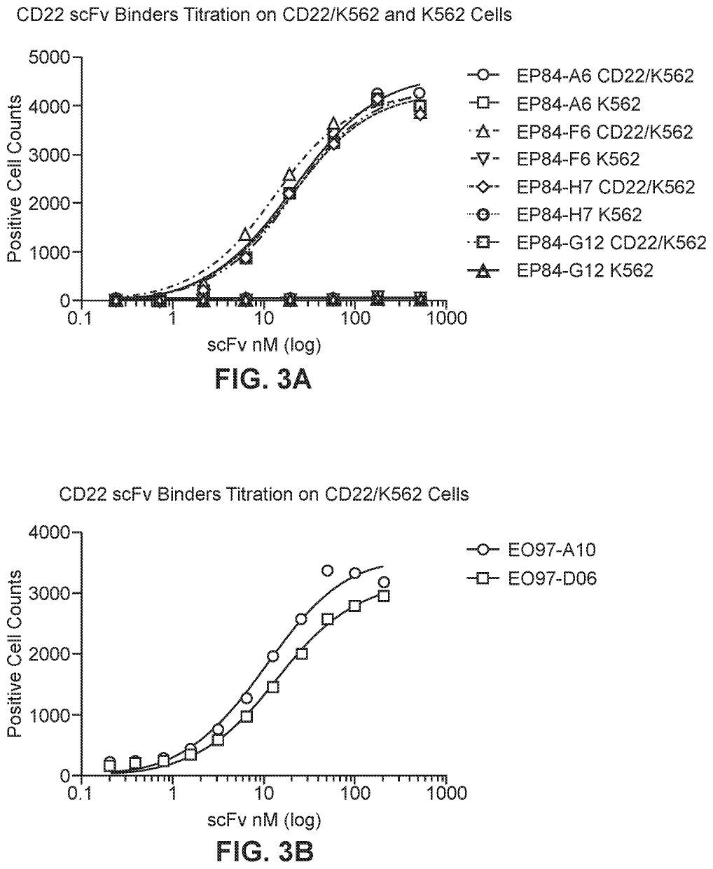

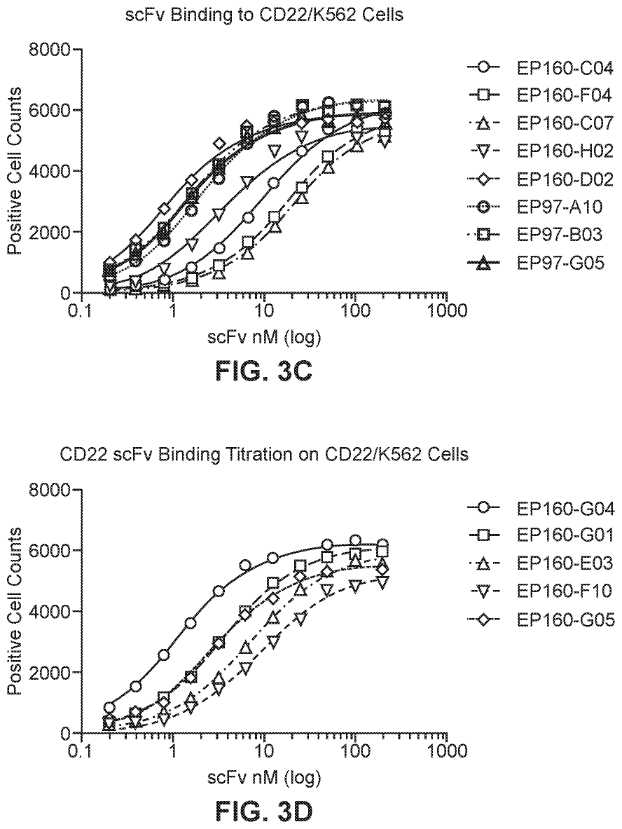

Exemplary anti-CD22 clones capable of binding to cell surface CD22 as determined in this study. FIGS. 3A-3D show binding curves of multiple exemplary anti-CD22 clones at various concentrations as indicated.

The binding affinities to CD22/K562 cells of a number of anti-CD22 antibodies disclosed herein are provided in Table 1 below:

TABLE 1
Binding Affinity of Exemplary Anti-
CD22 Antibodies to Cell Surface CD22

| Clone name: | EC₅₀ (nM) |
|---|---|
| EP160-D02 | 0.24 |
| EP160-H02 | 0.68 |
| EP97-B03 | 0.7 |
| EP97-A10 | 1 |
| EP160-G04 | 1.1 |
| EP160-F04 | 2.79 |
| EP160-G05 | 2.9 |
| EP97-G05 | 3.3 |
| EP35-C8 | 4.6 |
| EP160-C07 | 5.2 |
| EP160-E03 | 6.8 |
| EP160-F10 | 9 |
| EP97-F01 | 10 |
| EP35-A7 | 10.38 |
| EP35-E7 | 11 |
| EP35-E6 | 14.18 |
| EP35-F6 | 15 |
| EP35-C6 | 19.31 |
| EP35-D6 | 47 |
| EP35-B5 | 77 |

### EXAMPLE 3

### Epitope Binning of Anti-CD22 Molecules With M971 and/or BL22

### Epitope Binning of Anti-CD22 scFv Antibodies With M971

Epitope binning assay was performed to study whether any of the CD22 binders identified herein as disclosed in the above Examples can compete against M971, an anti-CD22 antibody, from binding to CD22. Briefly, CD22 overexpressing recombinant K562 cells were incubated either with 200 nM of a purified anti-CD22 scFv antibody disclosed herein or a pre-mixture containing 200 nM of the purified anti-CD22 scFv and 20 nM of M971 IgG antibody on ice for 1 hour. Cells were spun down at 1200 rpm for 5 minutes at 4° C. The binding activity of anti-CD22 scFv to the CD22-expressing K562 cells was included with an Alexa fluor 647-conjugated anti-His antibody (100 uL, 1:1000 dilution) at 4° C. for 30 minutes in the dark. The mixture thus formed was spun down at 1200 rpm for 5 minutes at 4° C. and washed twice with 200 uL of 1×PBS per well. The cells thus collected were reconstituted in 200 uL of 1×PBS and read on Attune flow cytometer. Analysis was performed by overlapping the binding histogram of 200 nM anti-CD22 scFv antibody to CD22 overexpressing recombinant K562 cells vs. that of the pre-mixed 200 nM anti-CD22 scFv antibody with 20 nM M971 IgG antibody to the same recombinant cells. The results thus obtained show that none of the scFv antibodies tested, including EP160-G04, EP97-B03, EP160-H02, EP97-A10, EP160-E03, EP160-F04, EP97-A01, EP35-C6, EP160-F10, EP160-G05, EP160-007, EP35-E6, EP35-C8, and EP35-F07 competes against M971 from binding to cell surface CD22.

Epitope binning with M971 was further confirmed by an ELISA assay. In brief, a 384-well plate was coated with 2 μg/mL of recombinant human CD22 or recombinant human Fc overnight at 4° C. The plate was then blocked with the Pierce superblock buffer for 1 hour at room temperature. 200 nM of a purified anti-CD22 scFv antibody as disclosed herein or a pre-mixture containing 200 nM of the purified anti-CD22 scFv and 100 nM of the M971 IgG antibody were loaded into the plate that was pre-coated with recombinant human Fc or recombinant human CD22. The plate was then incubated at room temperature for 1 hour with shaking. Afterwards, 25 uL of an HRP-conjugated anti-flag antibody (at 1:5000 dilution) was added to each well and the plate was incubated in dark at room temperature for one hour. The plate was washed for 3 times with 80 uL of 1×PBST in between each step. Afterwards, the plate was developed with 20 uL of the 1-step ultra TMB-ELISA substrate solution for five minutes, followed by adding 20 μL of 2N sulfuric acid to stop the reaction. The plate was read at OD₄₅₀ on a Biotek plate reader. Analysis were performed by graphing on Excel bar graph comparing the binding of 200 nM anti-CD22 scFv antibody only vs. pre-mixed 200 nM anti-CD22 scFv with 100 nM IgG M971 antibody on the plate of recombinant human CD22 protein.

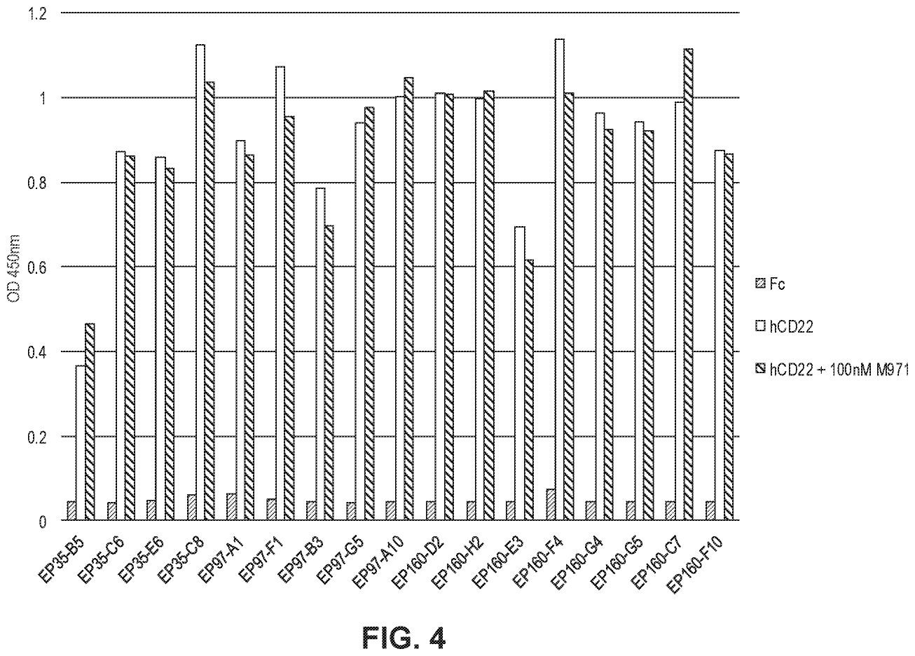

As shown in FIG. 4, none of the exemplary anti-CD22 scFv antibodies as indicated competes against M971 from binding to CD22.

### Anti-CD22 Antibody Binding Epitope Compared to M971 and BL22

BL22 (also known as CAT-3888) is a recombinant anti-CD22 immunotoxin proposed as a therapeutic for the treatment of B cell malignancies, and is known in the art. BL22 is a recombinant fusion protein comprising disulfide linked V_{H} and V_{L} chains of the mouse anti-CD22 monoclonal antibody RFB4 fused to a truncated form of Pseudomonas exotoxin A, termed PE38. Epitope specificity and tissue reactivity of RFB4 is reported in Li et al., Cell Immunol. 118(1):85-99 (1989).

CD22 EP160-D02 antibody epitope binning with M971 and BL22 was done by FACS analysis with EP160-D02 scFv and CD22 overexpressing recombinant K562 cell line. Purified anti-CD22 scFv was 2 fold serial diluted from 200 nM and pre-mixture of 5.13 nM, 1.77 nM of M971 or 0.7 nM, 0.175 nM of BL22 mAbs respectively on ice for one hour. Cells were spun down at 1200 rpm for 5 minutes at 4° C. The binding activity of anti-CD22 scFv was detected by anti-His Alexa fluor 647 by adding 100 uL of 1:1000 diluted secondary antibody and incubated at 4° C. for 30 minutes in the dark. Samples were spun down at 1200 rpm for 5 minutes at 4° C. and washed twice with 200 uL of 1×PBS per well. Cells were reconstituted in 200 uL of 1×PBS and read on Attune flow cytometer. Analysis was done by counting the anti-CD22 scFv positive staining cells on CD22 overexpressing recombinant K562 cells in the presence and absence of M971 and BL22 mAbs. EC₅₀ was calculated using Prism 8.0.

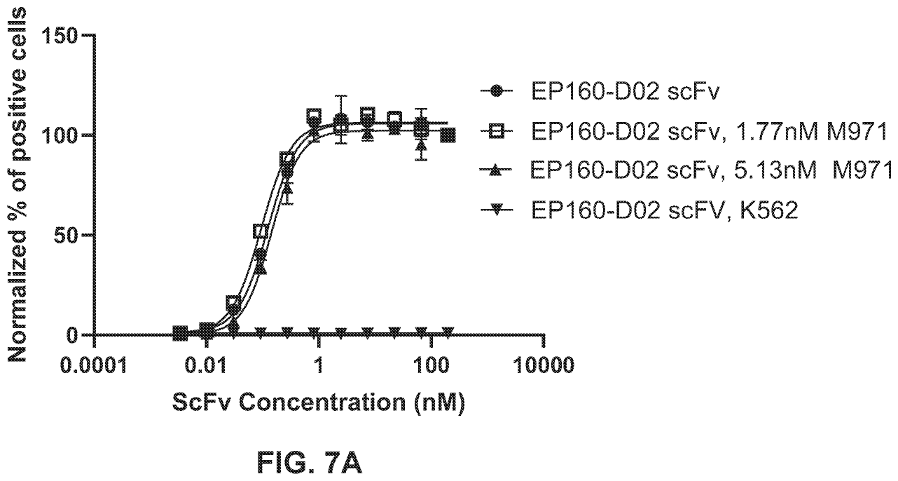

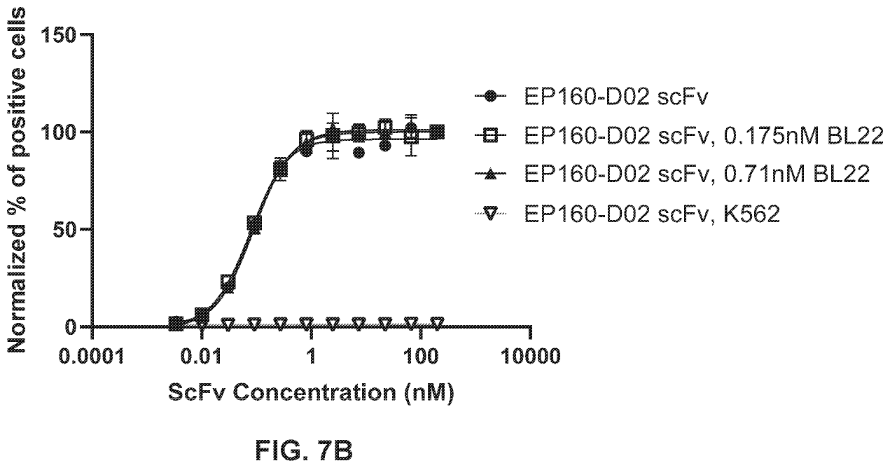

As shown in FIGS. 7A and 7B, presence of M971 and BL22 did not have significant impact on the binding activity of clone EP160-D02 to CD22-expressing K562 cells, indicating that M971 and BL22 do not compete with EP160-D02 for binding to cell surface CD22. In other words, the results show that EP160-D02 does not bind the same epitope as either M971 or BL22. The EC₅₀ and IC₅₀ values of EP160-D02 determined in this assay are provided in Tables 2 and 3 below:

TABLE 2
EC₅₀ Value of EP160-D02 in the Presence or Absence of M971

| | EC₅₀ (nM) |
|---|---|
| EP160-D02 scFv | 0.1246 |
| EP160-D02 scFv + 1.77 nM M971 | 0.09457 |
| EP160-D02 scFv + 5.13 nM M971 | 0.1436 |
| EP160-D02 scFv, K562 | N/D |

TABLE 3
IC₅₀ Value of EP160-D02 in the Presence or Absence of BL22

| | IC₅₀ (nM) |
|---|---|
| EP160-D02 scFv | 0.08216 |
| EP160-D02 scFv, 0.175 nM BL22 | 0.0811 |
| EP160-D02 scFv, 0.77 nM BL22 | 0.08978 |
| EP160-D02 scFv, K562 | N/D |

In sum, the results from these epitope binning assays indicate that the exemplary anti-CD22 antibodies reported herein (e.g., EP160-D02) do not bind to the same CD22 epitope as compared with known anti-CD22 antibodies M971 and R1-134. As such, the exemplary anti-CD22 antibodies disclosed herein would be expected to have different bioactivities in at least some aspects relative to the known anti-CD22 antibodies.

### EXAMPLE 4

### Binding Kinetics of Anti-CD22 scFv Antibodies

Kinetic analysis of the binding of anti-CD22 scFvs to CD22 have been assessed by the SPR technology with Biacore T200. The assay was run with Biacore T200 control software version 2.0. For each cycle, 1 μg/mL of human CD22-Fc fusion protein was captured for 60 seconds at flow rate of 10 ul/min on flow cell 2 in 1×HBST buffer on Protein G sensor chip. 2-fold serial diluted HIS tag purified anti-CD22 scFv was injected onto both reference flow cell 1 and CD22 captured flow cell 2 for 150 seconds at flow rate of 30 u1/mins followed by wash for 300 seconds. The flow cells were then regenerated with Glycine pH 2 for 60 seconds at flow rate of 30 ul/mins. 8 concentration points from 100-0 nM was assayed per anti-CD22 scFv in a 96 well plate. The kinetics of scFvs binding to CD22 protein was analyzed with Biacore T200 evaluation software 3000. The specific binding response unit was derived from subtraction of binding to reference flow cell 1 from CD22 captured flow cell 2. The results are provided in Table 4 below.

TABLE 4
Binding Kinetics of Exemplary Anti-CD22 scFv Antibodies

| scFv Clones | Ka (1/Ms) | Kd (1/s) | KD (M) |
|---|---|---|---|
| EP160-D02 | 5.51E+06 | 1.07E−04 | 1.94E−11 |
| EP97-G05 | 2.52E+05 | 4.19E−05 | 1.66E−10 |
| EP97-F01 | 8.88E+05 | 1.95E−04 | 2.19E−10 |
| EP160-G04 | 1.25E+05 | 4.03E−05 | 3.22E−10 |
| EP97-B03 | 3.76E+05 | 3.18E−04 | 8.46E−10 |
| EP160-H02 | 6.09E+04 | 1.12E−04 | 1.84E−09 |
| EP97-A10 | 4.05E+04 | 1.45E−04 | 3.57E−09 |
| EP160-E03 | 2.97E+05 | 1.64E−03 | 5.52E−09 |
| EP160-F04 | 4.05E+04 | 2.83E−04 | 5.89E−09 |
| EP35-F07 | 8.69E+04 | 9.80E−04 | 1.13E−08 |
| EP97-A01 | 7.13E+04 | 1.01E−03 | 1.42E−08 |
| EP35-C06 | 6.25E+04 | 9.85E−04 | 1.58E−08 |

### EXAMPLE 5

### Thermal Stability Assessment of Exemplary Anti-CD22 scFv Antibodies

In this example, each sample and control were prepared in at least a duplicate to make sure the results were reproducible. A plate map was designed first in Excel so the exact location of each sample can be matched to the software for running and analyzing the samples.

A fresh dilution of Protein Thermal Shift Dye (1000×) to 8× was prepared in water. A MicroAmp Optical 96 well plate or 8 cap strip by LifeTech were used for the experiments. The following reagents were added in the order listed:
1^{st} sample: 5 ul Protein Thermal Shift Buffer,
2^{nd} sample: 12.5 ul sample diluted to 0.4 mg/mL in water,
3^{rd} sample: 2.5 ul diluted Thermal Shift Dye 8× for a total volume of 20 ul/well.
Negative control sample: 12.5 ul buffer with no protein
Positive control sample: 10.5 ul water with 2.0 uL Protein Thermal Shift Control Protein.

The Thermal shift dye, once added, was pipetted up and down for 10 times. The plates or strips were then spun down for 1000 RPM for 1 min once sealed with MicroAmp Optical film of caps. Afterwards, the plate or strips was put into a Quant Studio 3 instrument by Thermo Fisher with the proceeding method being run as follows.
Step 1: 100% ramp rate to 25.0° with time 2 min
Step 2: 1% ramp rate to 99.0° C. with time 2 min

The samples and subsequent Tm were then analyzed (and Tm calculated) using the QuantStudio Design and Analysis Software and the Protein Thermal Shift Software 1.3. The results are shown in Table 5 below:

TABLE 5
Thermal Shift Assay of Exemplary Anti-CD22 Antibodies

| scFvs | Tm ° C. |
|---|---|
| EP160-D02 | 57.5 |
| EP97-G05 | 56.6 |
| EP97-F01 | 59.8 |
| EP160-G04 | 52.0 |
| EP97-B03 | 54.4 |
| EP160-H02 | 57.7 |
| EP97-A10 | 61.8 |
| EP160-E03 | 71.4 |
| EP160-F04 | 47.2 |
| EP35-F07 | 56.3 |
| EP97-A01 | 72.3 |
| EP35-C06 | 71.2 |
| EP35-B05 | 66.7 |
| EP160-F10 | 69.7 |
| EP160-G05 | 59.7 |
| EP160-C07 | 48.5 |
| EP35-C08 | 52 |

### EXAMPLE 6

### Anti-CD22 Antibodies Bind to Endogenous CD22 and Recombinant CD22 on the Cell Surface

Exemplary anti-CD22 scFv antibodies, including EP97-G05, EP97-A10, EP160-E03, and EP160-H02, were tested for their ability to bind to endogenous CD22 expressed on cell surface and recombinant CD22 expressed on cell surface using FACS analysis.

Briefly, 200 nM of each of purified CD22 scFv antibodies (containing a HIS tag) were diluted in full medium and incubated with Daudi, Raji, CD22/HEK293, CD22/K562, and K562 cell lines in 96 wells plate on ice for 1 hour. Cells were spun down at 1200 rpm for 5 minutes at 4° C. to remove unbound scFvs. Cells were then washed once with 200 uL of full medium per well. Samples were detected with anti-HIS biotin/Streptavidin Alexa® fluor 647 by adding 100 uL of diluted secondary antibody and incubated at 4 C for 30 minutes in the dark. Samples were spun down at 1200 rpm for 5 minutes at 4° C. and washed twice with 200 uL of 1×PBS per well. The samples were reconstituted in 200 uL of 1×PBS and read on Attune N×T cytometer. Analysis was done by Attune N×T software plotting the overlay histogram of CD22 proteins binding onto both negative and target cell lines. Anti-CD22 mouse antibody and anti-HIS biotin/Streptavidin secondary Alexa fluor 647 as positive and negative (background) control for the assay.

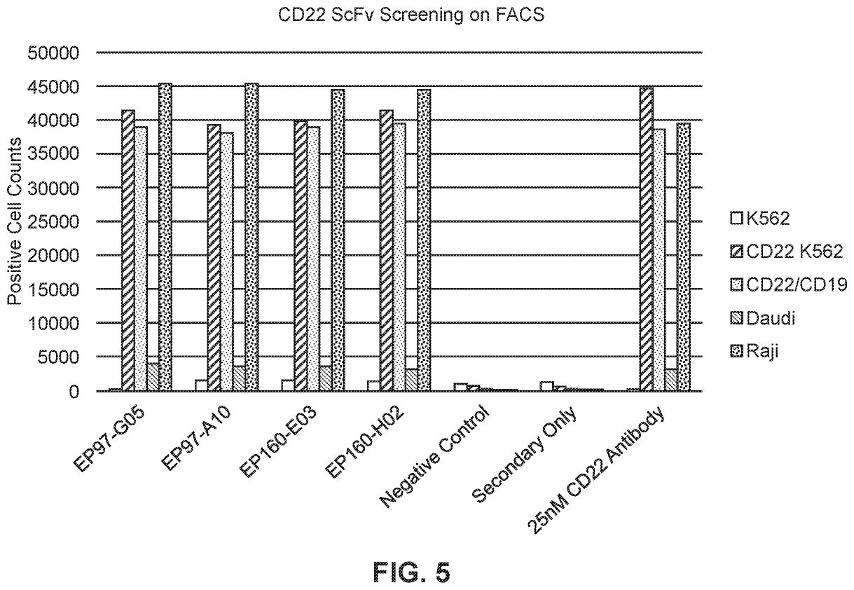

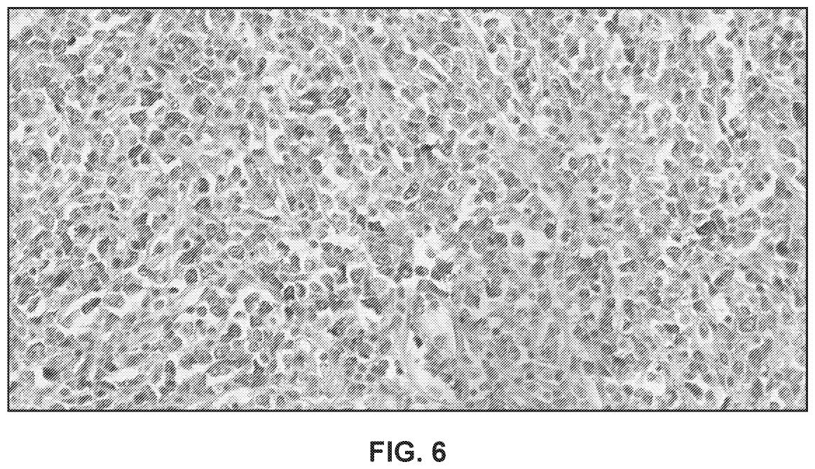

As indicated in FIG. 5, all four anti-CD22 scFv antibodies bind to HEK and K562 cells expressing recombinant CD22 on cell surface at the tested antibody concentration. The anti-CD22 scFvs were also found to bind to Daudi and Raji, which express endogenous CD22 on cell surface.

Further, immunohistochemistry (IHC) studies were performed with 5-mm sections from formalin-fixed, paraffin-embedded diffuse large B cell lymphoma (DLBCL) FFPE tissue block performed on Ventana Ultra automation platform using IHC staining protocol. Briefly, after deparaffinization and rehydration, the antigen retrieval was performed with standard CC1 antibody retrieval (EDTA based antigen retrieval buffer, pH 9.0, Cat #950-500). The tissue permeabilized and washed with Ventana discovery wash, Cat #905-510 and discovery reaction buffer, Cat #950-300 between staining steps. Discovery inhibitor CM Cat #764-4307 and IHC/ICC IHC protein blocker (Invitrogen Cat #00-4952-54) pretreatment for non-specific staining were applied during staining.

Exemplary anti-CD22 scFv, EP97-G05, fused with human Fc polypeptide, was incubated with the tissue samples noted above at a concentration of 10 ug/ml for 60 min at 37° C., followed by incubation with Anti-Human IgG FC HRP antibody at 1/250 dilution (Abcam Cat # ab98624). Ventana ChromapDAB kit (Cat #760-159) was used for final IHC steps. All the sections were counterstained with hematoxylin, and the whole slide imaged by Aperio AT2 scanscope and image analysis with Indica labs CytoNuclear v1.6 Algorithms.

As shown in FIG. 7, EP97-G05 was found to bind to CD22-positive DLBCL tissue in this IHC study described herein, indicating that this antibody is capable of binding to endogenous CD22, which may be expressed on disease cells.

### EXAMPLE 7

### Preparation and Characterization of Anti-CD22 IgG Antibodies

### (i) Recombinant Production of Anti-CD22 IgG Antibodies

The anti-CD22 ScFv antibodies were converted to IgG format following routine practice. Briefly, the V_{H} and V_{L} sequences were fused to the constant domains of human IgGlk backbone. Genes were codon optimized for mammalian expression, synthesized and subcloned to pCDNA3.4 expression vector by Life Technologies. The antibodies were expressed transiently in ExpiHEK293-F cells in free style system (Invitrogen) according to standard protocol. The cells were grown for five days before harvesting. The supernatant was collected by centrifugation and filtered through a 0.2 μm PES membrane.

The Fc fusion agonist first was purified by MabSelect PrismA protein A resin (GE Health). The protein was eluted with 100 mM Gly pH2.5 plus 150 mM NaCl and quickly neutralized with 20 mM Tris-HCl pH 8.0 plus 300 mM NaCl.

The antibodies were then further purified by a Superdex 200 Increase 10/300 GL column The monomeric peak fractions were pooled and concentrated. The final purified protein has endotoxin of less than 10 EU/mg and kept in 1×PBS buffer.

### (ii) Anti-CD22 IgG Antibody Cell Binding Activities

EC₅₀ of anti-CD22 IgG to CD22 overexpression recombinant cell line was determined by FACS binding assay. Purified IgG was 2-fold serial diluted in full medium for 12 times. The diluted IgGs were incubated with 100,000 CD22 K562 cells per well in 96 wells plate on ice for one hour. Cells were spun down at 1200 rpm for 5 minutes at 4° C. to remove unbound antibodies. Cells were then washed once with 200 uL of full medium per well. BL22 was used as a positive control of the anti-CD22 antibody and CHO-K1 cells expressing CD123 (but not CD22) were used as a negative control.

Samples were detected with anti-hFc Alexa fluor 488 by adding 100 uL of 1:1000 diluted secondary antibody and incubated at 4° C. for 30 minutes in dark. Samples were spun down at 1200 rpm for 5 minutes at 4° C. and washed twice with 200 uL of 1×PBS per well. Reconstituted samples in 200 uL of 1×PBS and read on Guava EasyCyte. Analysis was done by counting only positive Alexa Fluor 488 cells and then plotted in Prism 8.1 software.

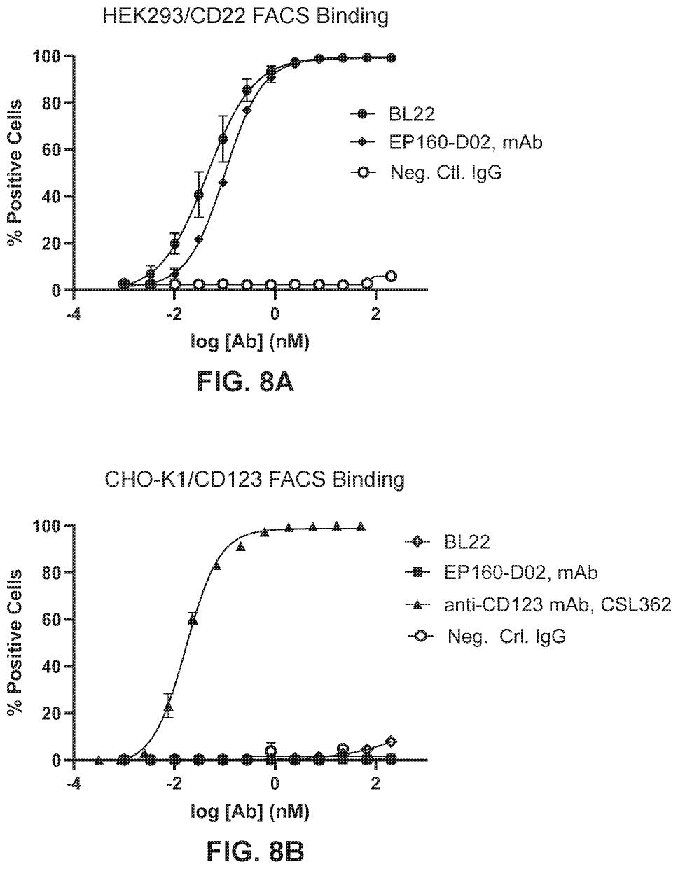

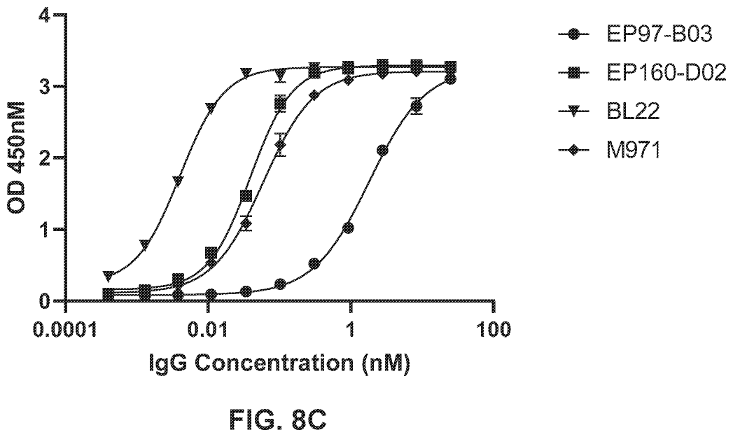

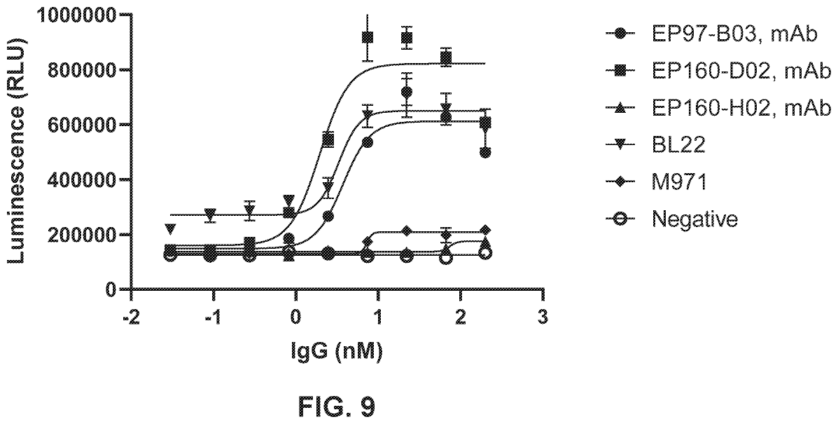

As shown in FIGS. 8A and 8B, clone EP160-D02 in IgG format showed strong binding to cell surface CD22, but no binding to cell surface CD123. The EC₅₀ values of the exemplary EP160-D2 (IgG) antibody are provided in Tables 6 and 7 below.

TABLE 6
EC₅₀ Value of Binding to Cell Surface CD22

| | HEK293-CD22 |
|---|---|
| Antibody | EC₅₀ (nM) |
| BL22 (anti-CD22) | 0.044 |
| EP160-D02 (IgG) | 0.101 |
| Neg (hIgG1k) | N.D. |

TABLE 7
EC₅₀ Value of Binding to Cell Surface CD123

| | CHOK1-CD123 |
|---|---|
| Antibody | EC₅₀ (nM) |
| BL22 (anti-CD22) | N.D. |
| CSL362 (anti-CD123) | 0.017 |
| Neg (hIgG1k) | N.D. |
| EP160-D02 (IgG) | N.D. |

Binding to cell surface CD22 by the anti-CD22 IgG antibodies was also determined by ELISA and similar results were observed. See FIG. 8C and Table 8 below.

TABLE 8
EC₅₀ Values of Anti-CD22 IgG Antibodies by ELISA

| | EC₅₀ (nM) |
|---|---|
| EP160-D02 | 0.039 |
| EP97-B03 | 1.82 |
| BL22 | 0.004 |
| M971 | 0.059 |

### (iii) Anti-CD22 IgG Antibody ADCC Activities

Antibody-dependent cellular cytotoxicity (ADCC), also referred to as antibody-dependent cell-mediated cytotoxicity, is a mechanism of cell-mediated cytotoxic process whereby an effector cell of the immune system is engaged by an antibody and actively lyses a target cell to which the antibody binds.

The ADCC activities of the anti-CD22 IgG antibodies were tested with Promega ADCC Bioreporter assay kit. Briefly, 30,000 CD22/HEK293 target cells were plated on white bottom flat 96 well assay plate and incubated at 37° C. overnight. Following manufacture's protocol, antibodies were 3-fold serial diluted from 200 nM in ADCC assay buffer. Supernatant from target cells was removed. 25 μL of ADCC assay buffer mixed with 25 μL of antibody dilution was added to each well of cells. Cells were incubated at room temperature for one hour before effector cells were added.

Effector cells were thawed following manufacture's protocol and 25 μL of effector cells was plated to each target cells/antibody mixture. The plate was incubated at 37 C for 16 hours.

The following day, samples were equilibrated at room temperature for 30 minutes and then 75 μL of room temperature Bio-glow reagent was added and incubated at room temperature shaking for 30 minutes in the dark. Bio-glow reagent was prepared according to the manufacturing protocol. The plate was read with luminescence on Biotek Neo2 plate reader and data was graphed on Prism 8.0.

The results obtained from this assay show that the exemplary anti-CD22 IgG antibodies, including EP97-B03 and EP160-D02, exhibited ADCC activity, while control antibody M971 exhibited little or no ADCC activity. At least clone EP160-D02 showed better ADCC activity relative to BL22. The EC₅₀ values of the tested antibodies are provided in Table 9 below:

TABLE 9
EC₅₀ of Anti-CD22 Antibodies in ADCC Assay

| | EC₅₀ (nM) |
|---|---|
| EP97-B03 | 3.714 |
| EP160-D02 | 1.947 |
| EP160-H02 | ~73.80 |
| BL22 | 3.314 |
| M971 | ~ |

### (iv) Anti-CD22 IgG Antibody Internalization Activities

The kinetics of anti-CD22 antibody internalization was determined with image-based fluorescence assay. Briefly, 30,000 CD22/HEK293 target cells were plated on poly-L-lysine treated 96 well black bottom plate and incubated at 37° C. overnight. CD22 IgG and secondary antibody pHrodo were diluted to a final concentration of 4 nM and 120 nM, respectively, in 10% RPMI without phenol red and incubated at room temperature for a minimum of five minutes in the dark.

Medium was then removed from target cells and 100 μL of antibody/secondary pHrodo mixture was added to the cells. Cells were imaged with RFP and bright field on Cytation 5 immediately and at every two hours at 37° C. The rate of internalization was quantified by Cytation 5 analysis software and analyzed by Prism 8.0.

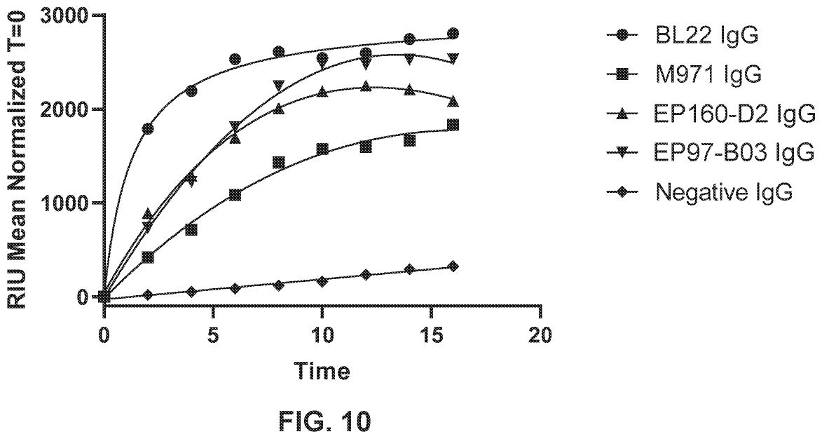

As shown in FIG. 10, clones EP160-D02 and EP97-B03 showed cellular internalization, albeit slower than the internalization of the BL22 molecule. See also Table 10 below.

TABLE 10
Internationalization of Anti-CD22 IgG Antibodies

| | T½ (Hour) |
|---|---|
| BL22 | 3.95 |
| M971 | 6.07 |
| EP160-D02 | 5.06 |
| EP97-B03 | 5.26 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An isolated antibody that binds CD22, wherein the antibody binds to the same epitope as a reference antibody or competes against the reference antibody from binding to CD22, and wherein the reference antibody is selected from the group consisting of EP35-A7, EP35-B05, EP35-C6, EP35-C8, EP35-D6, EP35-E6, EP35-E7, EP97-A01, EP97-A10, EP97-B03, EP97-F01, EP97-G05, EP160-007, EP160-D02, EP160-E03, EP160-F04, EP160-F10, EP160-G04, EP160-G05, and EP160-H02.

2. The isolated antibody of claim 1, wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

3. The isolated antibody of claim 1 or claim 2, wherein the HC CDRs of the antibody collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

4. The isolated antibody of any one of claims 1-3, wherein the antibody comprises a V_{H} that is at least 85% identical to the V_{H} of the reference antibody, and/or a V_{L} that is at least 85% identical to the V_{L} of the reference antibody.

5. The isolated antibody of any one of claims 1-4, wherein the antibody has a binding affinity of less than 10 nM to CD22 expressed on cell surface.

6. The isolated antibody of claim 5, wherein the antibody has a binding affinity of less than 1 nM to CD22 expressed on cell surface.

7. The isolated antibody of claim 1, which comprises the same heavy chain complementary determining regions (HC CDRs) and the same light chain complementary determining regions (LC CDRs) as the reference antibody.

8. The isolated antibody of claim 7, which comprises the same V_{H} and the same V_{L} as the reference antibody.

9. The isolated antibody of any one of claims 1-8, wherein the antibody is a human antibody or a humanized antibody.

10. The isolated antibody of any one of claims 1-9, wherein the antibody is a full-length antibody or an antigen-binding fragment thereof.

11. The isolated antibody of any one of claims 1-9, wherein the antibody is a single-chain antibody (scFv).

12. The isolated antibody of claim 11, wherein the antibody comprises an amino acid sequence selected the group consisting of SEQ ID NOs: 40-59.

13. A nucleic acid or a set of nucleic acids, which collectively encodes the antibody of any one of claims 1-12.

14. The nucleic acid or the set of nucleic acids of claim 13, which is a vector or a set of vectors.

15. The nucleic acid or the set of nucleic acids or claim 14, wherein the vector is an expression vector.

16. A host cell comprising the nucleic acid or the set of nucleic acids of any one of claims 13-15.

17. A pharmaceutical composition comprising the antibody of any one of claims 1-12, the nucleic acid or nucleic acids of any one of claims 13-15, or the host cell of claim 16, and a pharmaceutically acceptable carrier.

18. A method for inhibiting CD22 in a subject, comprising administering to a subject in need thereof any effective amount of the pharmaceutical composition of claim 17.

19. The method of claim 18, wherein the subject is a human patient having CD22 positive disease cells.

20. The method of claim 18 or claim 19, wherein the subject is a human patient having cancers or an autoimmune diseases.

21. The method of claim 20, wherein the human patient has CD22 positive cancer cells or CD22 positive auto-reactive immune cells.

22. A method for detecting presence of CD22, comprising:
(i) contacting an antibody of any one of claims 1-12 with a sample suspected of containing CD22, and
(ii) detecting binding of the antibody to CD22.

23. The method of claim 22, wherein the antibody is conjugated to a detectable label.

24. The method of claim 22 or claim 23, wherein the CD22 is expressed on cell surface.

25. The method of any one of claims 22-24, wherein the contacting step is performed by administering the antibody to a subject.

26. A method of producing an antibody binding to CD22, comprising:
(i) culturing the host cell of claim 16 under conditions allowing for expression of the antibody that binds CD22; and
(ii) harvesting the antibody thus produced from the cell culture.


SEQUENCE LISTING (60 sequences)

SEQ ID NO: 1 - protein, 123 aa, Artificial Sequence
  Description of Artificial Sequence Synthetic polypeptide
QVQLQQSGPGLVKPSQTLSLTCAISGDSVSSNSAAWNWIRQSPSRGLEWLGRTYYRSWYNDYAVSVKSRITINPDTSKNQFSLQLNSVTPEDTAVYYCAREVTGDLEDAFDIWGQGTMVTVSS

SEQ ID NO: 2 - protein, 107 aa, Artificial Sequence
  Description of Artificial Sequence Synthetic polypeptide
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 3 - protein, 118 aa, Artificial Sequence
  Description of Artificial Sequence Synthetic polypeptide
QMQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDAVAGSRGYWGQGTLVTVSS

SEQ ID NO: 4 - protein, 108 aa, Artificial Sequence
  Description of Artificial Sequence Synthetic polypeptide
EIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGEGTKVEIK

SEQ ID NO: 5 - protein, 117 aa, Artificial Sequence
  Description of Artificial Sequence Synthetic polypeptide
EVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSS

SEQ ID NO: 6 - protein, 108 aa, Artificial Sequence
  Description of Artificial Sequence Synthetic polypeptide
EIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 7 - protein, 121 aa, Artificial Sequence
  Description of Artificial Sequence Synthetic polypeptide
EVQLVESGGGVVQPGRSLRLSCVASGFTFRNYGMQWVRQTPDKGLEWVAVTAHDGTVQYYVDSVKGRFTISRDNSKDTLYLQMNSLRVADTAVYYCAKEATPRAADHFDYWGQGTLGTVSS

SEQ ID NO: 8 - protein, 121 aa, Artificial Sequence
  Description of Artificial Sequence Synthetic polypeptide
QVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSS

SEQ ID NO: 9 - protein, 108 aa, Artificial Sequence
  Description of Artificial Sequence Synthetic polypeptide
EIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIK

SEQ ID NO: 10 - protein, 113 aa, Artificial Sequence
  Description of Artificial Sequence Synthetic polypeptide
QLVQSGAEVKKPGASVKVSCKASGYTFSSYGITWVRQAPGQGLEWMGWISAYNGNTNYAQKFQGRVTLTTDTSTSIAYMELRSLTSDDTAVYYCATGGQEDYWGQGTLVTVSS

SEQ ID NO: 11 - protein, 108 aa, Artificial Sequence
  Description of Artificial Sequence Synthetic polypeptide
EIVLTQSPATLSVSPGERATLSCRASQSVSSNLAWYQQKPGQAPRLLIYGASTRATGIPARFSGSGSGTEFTLTISSLQSEDFAVYYCQQYNSWPPLTFGGGTKVEIK

SEQ ID NO: 12 - protein, 119 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDPLEPLESDYWGQGTLVTVSS

SEQ ID NO: 13 - protein, 108 aa, Artificial Sequence
  Description of Artificial Sequence Synthetic polypeptide
EIVMTQSPATLSVSPGEGVTLSCRASQSVSSNLAWYQQRPGQAPRLLIYGASIKATDVPDRFSGGGSGTDFSLSITNLQSEDFAVYYCQQYHTWPPVTFGGGTKVEIK

SEQ ID NO: 14 - protein, 115 aa, Artificial Sequence
  Description of Artificial Sequence Synthetic polypeptide
QVQLVQSGAEVKKPGASVKVSCKASGYTFSSYGITWVRQAPGQGLEWMGWISAYNGNTNYAQKFQGRVTLTTDTSTSIAYMELRSLTSDDTAVYYCATGGQEDYWGQGTLVTVSS

SEQ ID NO: 15 - protein, 108 aa, Artificial Sequence
  Description of Artificial Sequence Synthetic polypeptide
EIVMTQSPATLSVSPGERATLSCRASQSVSSNLAWYQQKPGQAPRLLIYDASTRATGIPARFSGSGSGTEFTLTISSLQSEDFAVYYCQQYHNWAPLTFGGGTKVGIK

SEQ ID NO: 16 - protein, 122 aa, Artificial Sequence
  Description of Artificial Sequence Synthetic polypeptide
QVQLVQSGAEVKKPGASVKVSCKASGYTFTSYGISWVRQAPGQGLEWMGGIIAYNGNTNYAQKLQGRVTMTTDTSTSTAYMELRSLRSDDTAVYYCARDPPEYSSSAGTDYWGQGTLVTVSS

SEQ ID NO: 17 - protein, 108 aa, Artificial Sequence
  Description of Artificial Sequence Synthetic polypeptide
EIVMTQSPATLSVSPGEGVTLSCRASQSVSSNLAWYQQRPGQAPRLLIYGASIKATDVPDRFSGGGSGTDFTLSITNLQSEDFAVYYCQQYHTWSPVTFGGGTKVEIK

SEQ ID NO: 18 - protein, 115 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDPSMDVWGQGTTVTVSS

SEQ ID NO: 19 - protein, 108 aa, Artificial Sequence
  Description of Artificial Sequence Synthetic polypeptide
EIVLTQSPATLSVSPGERATLSCRASQSVSSNLAWYQQKPGQAPRLLIYGASTRATGIPARFSGSGSGTEFTLTISSLQSEDFAVYYCQQYNSWPPITFGQGTRLEIK

SEQ ID NO: 20 - protein, 123 aa, Artificial Sequence
  Description of Artificial Sequence Synthetic polypeptide
QVQLVESGGGVVQPGGSLRLSCAASGFPFSRFGIHWVRQAPGKGLDWVAFIRTDGGSQHYADSVKGRFTISRDNSENMVYLQMNSLRVDDTALYYCAKDPPRVTGNTGYDYDWGQGVQVTVSS

SEQ ID NO: 21 - protein, 113 aa, Artificial Sequence
  Description of Artificial Sequence Synthetic polypeptide
DIVMTQSPDSLAVSLGERATINCKSSQSVLYSANNKNCLAWYQQKSGQPPKLLIYWASTRESGVPGRFSGSGSGTDFTLTISSLQAEDVAVYYCQQYYSPPRTFGQGTKLEIK

SEQ ID NO: 22 - protein, 122 aa, Artificial Sequence
  Description of Artificial Sequence Synthetic polypeptide
EVQLVESRGGVVQPGGSLRLSCAASGFTFSSYGMHWVRQAPGKGLEWVAFIRYDGSNKYYADSVKGRFTISRDNSKNTLYLQMNSLRAEDTAVYYCAKETVTTNYYYYMDVWGKGTTVTVSS

SEQ ID NO: 23 - protein, 112 aa, Artificial Sequence
  Description of Artificial Sequence Synthetic polypeptide
DVVMTQSPLSLPVTLGQPASISCRSSRSLEYNDGNTYLNWFHQRPGQSPRRLIYKVSNRDSGVPDRFSGSGSDTDFTLKISRVEAEDVGIYYCMQGTHWPLTFGQGTRLEIK

SEQ ID NO: 24 - protein, 119 aa, Artificial Sequence
  Description of Artificial Sequence Synthetic polypeptide
QVQLVQSGTEVKKPGASVKVSCKASGYTFTNNAITWVRQAPGQGLEWMGYISTSSDNINYAQKFRGRLTLTTDTSTGTAYMELSSLRSDDTATYYCARDGIFGGRDDPWGQGTLVTVSS

SEQ ID NO: 25 - protein, 105 aa, Artificial Sequence
  Description of Artificial Sequence Synthetic polypeptide
DIVMTQSPSSLSASVGDRVTITCQASQDISNYLNWYQQKPGKAPKLLIYDASNLETGVPSRFSGSGSETDFTITISSLQPEDIATYYCQQYDNLPLTFGGGTKVR

SEQ ID NO: 26 - protein, 113 aa, Artificial Sequence
  Description of Artificial Sequence Synthetic polypeptide
QVQLVESGGALVQPGGSLRLSCVVSGFPFSTAWMNWVRQAPGKGLEWVARIKSEAHGGTTHYAPPVQGRFTISRDDSKNTVSLQMNSLKTEDTGVYYCGDFQWGQGTLVTVSS

SEQ ID NO: 27 - protein, 107 aa, Artificial Sequence
  Description of Artificial Sequence Synthetic polypeptide
VIWMTQSPSSLSASVGDRITITCQASQDISNFLNWYQQKPGEAPKLLLYDASNLERGVPSRFSGGGSGTDFTLTISSLQPEDIATYFCQQYDNLPLTFGGGTKVEIK

SEQ ID NO: 28 - protein, 118 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLGSDDTAVYYCARDSGSSDLDYWGQGTLVTVSS

SEQ ID NO: 29 - protein, 108 aa, Artificial Sequence
  Description of Artificial Sequence Synthetic polypeptide
EIVMTQSPATLSVSPGEGVTLSCRASQSVSSNLAWYQQKPGQAPRLLMYGASIKATDVPDRFSGGGSGTDFTLSISSLQSEDFAVYYCQQYHTWPPVTFGGGTKVEIK

SEQ ID NO: 30 - protein, 116 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLKSDDTAVYYCAISIGAFDIWGQGTMVTVSS

SEQ ID NO: 31 - protein, 108 aa, Artificial Sequence
  Description of Artificial Sequence Synthetic polypeptide
EIVMTQSPATLSVSPGEEVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 32 - protein, 118 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSGNSPIDYWGQGTLVTVSS

SEQ ID NO: 33 - protein, 108 aa, Artificial Sequence
  Description of Artificial Sequence Synthetic polypeptide
EIVMTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 34 - protein, 119 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSS

SEQ ID NO: 35 - protein, 108 aa, Artificial Sequence; identical to SEQ ID NO: 6
  Description of Artificial Sequence Synthetic polypeptide

SEQ ID NO: 36 - protein, 118 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDHIAAAGDYWGQGTLVTVSS

SEQ ID NO: 37 - protein, 108 aa, Artificial Sequence
  Description of Artificial Sequence Synthetic polypeptide
EIVMTQSPATLSVSPGEGVTLSCRASQSVSSNLAWYQQRPGQAPRLLIYGASIKATDVPDRFSGGGSGTDFTLSITNLQSEDFAVYYCQQYHTWPPVTFGGGTKVEIK

SEQ ID NO: 38 - protein, 117 aa, Artificial Sequence
  Description of Artificial Sequence Synthetic polypeptide
EVQLVQSGGGVVQPGRSLKLSCAASGFTFSSYGMHWVRQAPGKGLEWVAVIWYDGSNKYYADSVKGRFTISRDNSKNTLYLQMNSLRAEDTAVYYCARDGWKGFDYWGQGTTVTVSS

SEQ ID NO: 39 - protein, 108 aa, Artificial Sequence; identical to SEQ ID NO: 6
  Description of Artificial Sequence Synthetic polypeptide

SEQ ID NO: 40 - protein, 241 aa, Artificial Sequence
  Description of Artificial Sequence Synthetic polypeptide
QMQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDAVAGSRGYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGEGTKVEIK

SEQ ID NO: 41 - protein, 240 aa, Artificial Sequence
  Description of Artificial Sequence Synthetic polypeptide
EVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 42 - protein, 121 aa, Artificial Sequence; identical to SEQ ID NO: 7
  Description of Artificial Sequence Synthetic polypeptide

SEQ ID NO: 43 - protein, 244 aa, Artificial Sequence
  Description of Artificial Sequence Synthetic polypeptide
QVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIK

SEQ ID NO: 44 - protein, 236 aa, Artificial Sequence
  Description of Artificial Sequence Synthetic polypeptide
QLVQSGAEVKKPGASVKVSCKASGYTFSSYGITWVRQAPGQGLEWMGWISAYNGNTNYAQKFQGRVTLTTDTSTSIAYMELRSLTSDDTAVYYCATGGQEDYWGQGTLVTVSSGGGGSGGGGSGGGGSEIVLTQSPATLSVSPGERATLSCRASQSVSSNLAWYQQKPGQAPRLLIYGASTRATGIPARFSGSGSGTEFTLTISSLQSEDFAVYYCQQYNSWPPLTFGGGTKVEIK

SEQ ID NO: 45 - protein, 242 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDPLEPLESDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFSLSITNLQSEDFAVYYCQQYHTWPPVTFGGGTKVEIK

SEQ ID NO: 46 - protein, 238 aa, Artificial Sequence
  Description of Artificial Sequence Synthetic polypeptide
QVQLVQSGAEVKKPGASVKVSCKASGYTFSSYGITWVRQAPGQGLEWMGWISAYNGNTNYAQKFQGRVTLTTDTSTSIAYMELRSLTSDDTAVYYCATGGQEDYWGQGTLVTVSSGGGGSGGGGSGGGGSEIVMTQSPATLSVSPGERATLSCRASQSVSSNLAWYQQKPGQAPRLLIYDASTRATGIPARFSGSGSGTEFTLTISSLQSEDFAVYYCQQYHNWAPLTFGGGTKVGIK

SEQ ID NO: 47 - protein, 245 aa, Artificial Sequence
  Description of Artificial Sequence Synthetic polypeptide
QVQLVQSGAEVKKPGASVKVSCKASGYTFTSYGISWVRQAPGQGLEWMGGIIAYNGNTNYAQKLQGRVTMTTDTSTSTAYMELRSLRSDDTAVYYCARDPPEYSSSAGTDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSITNLQSEDFAVYYCQQYHTWSPVTFGGGTKVEIK

SEQ ID NO: 48 - protein, 238 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDPSMDVWGQGTTVTVSSGGGGSGGGGSGGGGSEIVLTQSPATLSVSPGERATLSCRASQSVSSNLAWYQQKPGQAPRLLIYGASTRATGIPARFSGSGSGTEFTLTISSLQSEDFAVYYCQQYNSWPPITFGQGTRLEIK

SEQ ID NO: 49 - protein, 240 aa, Artificial Sequence
  Description of Artificial Sequence Synthetic polypeptide
EVQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCARDGWKGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 50 - protein, 251 aa, Artificial Sequence
  Description of Artificial Sequence Synthetic polypeptide
QVQLVESGGGVVQPGGSLRLSCAASGFPFSRFGIHWVRQAPGKGLDWVAFIRTDGGSQHYADSVKGRFTISRDNSENMVYLQMNSLRVDDTALYYCAKDPPRVTGNTGYDYDWGQGVQVTVSSGGGGSGGGGSGGGGSDIVMTQSPDSLAVSLGERATINCKSSQSVLYSANNKNCLAWYQQKSGQPPKLLIYWASTRESGVPGRFSGSGSGTDFTLTISSLQAEDVAVYYCQQYYSPPRTFGQGTKLEIK

SEQ ID NO: 51 - protein, 249 aa, Artificial Sequence
  Description of Artificial Sequence Synthetic polypeptide
EVQLVESRGGVVQPGGSLRLSCAASGFTFSSYGMHWVRQAPGKGLEWVAFIRYDGSNKYYADSVKGRFTISRDNSKNTLYLQMNSLRAEDTAVYYCAKETVTTNYYYYMDVWGKGTTVTVSSGGGGSGGGGSGGGGSDVVMTQSPLSLPVTLGQPASISCRSSRSLEYNDGNTYLNWFHQRPGQSPRRLIYKVSNRDSGVPDRFSGSGSDTDFTLKISRVEAEDVGIYYCMQGTHWPLTFGQGTRLEIK

SEQ ID NO: 52 - protein, 239 aa, Artificial Sequence
  Description of Artificial Sequence Synthetic polypeptide
QVQLVQSGTEVKKPGASVKVSCKASGYTFTNNAITWVRQAPGQGLEWMGYISTSSDNINYAQKFRGRLTLTTDTSTGTAYMELSSLRSDDTATYYCARDGIFGGRDDPWGQGTLVTVSSGGGGSGGGGSGGGGSDIVMTQSPSSLSASVGDRVTITCQASQDISNYLNWYQQKPGKAPKLLIYDASNLETGVPSRFSGSGSETDFTITISSLQPEDIATYYCQQYDNLPLTFGGGTKVR

SEQ ID NO: 53 - protein, 235 aa, Artificial Sequence
  Description of Artificial Sequence Synthetic polypeptide
QVQLVESGGALVQPGGSLRLSCVVSGFPFSTAWMNWVRQAPGKGLEWVARIKSEAHGGTTHYAPPVQGRFTISRDDSKNTVSLQMNSLKTEDTGVYYCGDFQWGQGTLVTVSSGGGGSGGGGSGGGGSVIWMTQSPSSLSASVGDRITITCQASQDISNFLNWYQQKPGEAPKLLLYDASNLERGVPSRFSGGGSGTDFTLTISSLQPEDIATYFCQQYDNLPLTFGGGTKVEIK

SEQ ID NO: 54 - protein, 241 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLGSDDTAVYYCARDSGSSDLDYWGQGTLVTVSSGGGGSGGGGSGGGGSEIVMTQSPATLSVSPGEGVTLSCRASQSVSSNLAWYQQKPGQAPRLLMYGASIKATDVPDRFSGGGSGTDFTLSISSLQSEDFAVYYCQQYHTWPPVTFGGGTKVEIK

SEQ ID NO: 55 - protein, 239 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLKSDDTAVYYCAISIGAFDIWGQGTMVTVSSGGGGSGGGGSGGGGSEIVMTQSPATLSVSPGEEVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 56 - protein, 241 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSGNSPIDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 57 - protein, 242 aa, Artificial Sequence
  Description of Artificial Sequence Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 58 - protein, 241 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDHIAAAGDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSITNLQSEDFAVYYCQQYHTWPPVTFGGGTKVEIK

SEQ ID NO: 59 - protein, 240 aa, Artificial Sequence
  Description of Artificial Sequence Synthetic polypeptide
EVQLVQSGGGVVQPGRSLKLSCAASGFTFSSYGMHWVRQAPGKGLEWVAVIWYDGSNKYYADSVKGRFTISRDNSKNTLYLQMNSLRAEDTAVYYCARDGWKGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 60 - protein, 6 aa, Artificial Sequence
  Description of Artificial Sequence Synthetic 6xHis tag
HHHHHH